(19) United States
(12) Patent Application Publication
(10) Pub. No.: US 2023/0407257 A1
(43) Pub. Date: Dec. 21, 2023

(54) TRANSFORMED NATURAL KILLER CELL

(71) Applicant: Purdue Research Foundation, West Lafayette, IN (US)

(72) Inventors: Sandro Matosevic, Lafayette, IN (US); Kyle Lupo, New York, NY (US)

(73) Assignee: Purdue Research Foundation, (omitted)

(21) Appl. No.: 18/177,023

(22) Filed: Mar. 1, 2023

Related U.S. Application Data
(60) Provisional application No. 63/315,650, filed on Mar. 2, 2022.

(51) Int. Cl. C12N 5/0783; C07K 16/28

(52) U.S. Cl. CPC C12N 5/0646; C07K 16/2896; C07K 16/2878; C12N 2506/03

(57) ABSTRACT

The present application generally relates to natural killer cells (NK) and genetically modified NK cells, methods of transformation, and methods of use in combination with other molecules for targeting cells of interest.

### CROSS REFERENCE TO RELATED APPLICATION

This present patent application relates to and claims the priority under 35 U.S.C. § 119(e) to U.S. Provisional Patent Application Ser. No. 63/315,650 filed on Mar. 2, 2022 the content of which is hereby incorporated by reference in its entirety into the present disclosure.

### GOVERNMENT SUPPORT CLAUSE

This invention was made with government support under a grant CA256413 awarded by the National Institutes of Health. The government has certain rights in the invention.

### SEQUENCE LISTING STATEMENT

A computer-readable form (CRF) of the Sequence Listing is submitted with this application. The file, entitled PRF69771-02.xml (19 kb), is generated on Feb. 28, 2023. Applicant states that the content of the computer-readable form file is the same to the written sequence listing as shown in the Figures. The content of the sequence listing file PRF69771-02.xml is hereby incorporated by reference in the entirety into this specification.

### TECHNICAL FIELD

The present application relates to a genetically engineered pluripotent stem cell derived natural killer cell (iNK), and more generally to methods and compositions for modifying Natural Killer Cell (NK) activity; and in more particularity for modifying NK cell inhibition when interacting with cells expressing CD155, and/or additionally other cell surface molecules such as CD73 and/or CD137, such as found on glioblastoma multiforme cells.

### BACKGROUND

This section introduces aspects that may help facilitate a better understanding of the disclosure. Accordingly, these statements are to be read in this light and are not to be understood as admissions about what is or is not prior art.

Cancer is a cellular disease state that is a prime target for therapeutic intervention and a highly active area of research. Methods and materials for characterizing cancer cells and their interactions are critical for developing and discovering and implementing therapeutic strategies and medicines. For example, glioblastoma multiforme (GBM) is one of the world's deadliest cancers, with very few available treatments. Patients diagnosed with GBM have a median of less than fifteen months, and nearly fifty percent of patients are unresponsive to current chemotherapeutic treatments. GBM cells are known to express CD155 cell surface protein which interacts with TIGIT (T-cell immunoglobulin and ITIM domain) on natural killer cells leading to many subsequent effects. TIGIT, a member of the Ig super family and an immune inhibitory receptor, plays a key role in the suppression of T-cell proliferation and activation; it is involved in tumor cell immune evasion, and the inhibition of antiviral immune responses.

NK cells (Natural Killer cells) can specifically target cancer cells, mitigating damage to healthy cells, and can be engineered to overcome cancer-induced immunosuppression and enhance their effector function. However, the complexity of cell interactions and responses have continued to be a barrier to understanding.

In view of the above, it would be useful to have transformed NK cells which target the TIGIT/CD155 interaction and allows for subsequent activation and release of additional effector molecules which target other cell surface receptors. It would be advantageous to have NK cells which maintain activity in the microenvironment of a target cell, including tumor microenvironments. An object of this disclosure is to provide a transformed NK cell which is derived from a transformed pluripotent stem cell such that the NK cell specifically targets the TIGIT/CD155 interaction and provides for activation of other effector molecules. It is an additional object of this disclosure to provide an operative transformed NK cell that targets the TIGIT/CD155 interaction and provides for the subsequent activation of an anti-CD73 effector molecule. An object of the present disclosure is to provide for a novel engineered cell and methods of altering the NK cell inhibition by subsequent target cells, by coadministration of the transformed TIGIT/CD155 NK cells with blockade of CD137 (41BB) by an additional effector molecule. This and other objects and advantages, as well as inventive features, will be apparent from the detailed description provided herein.

### SUMMARY

The present application relates generally to a natural killer cell which is derived from a pluripotent stem cell, stability transformed with a synthetic notch receptor protein construct, said construct comprising at minimum, nucleic acid domain segments which encode for, a TIGIT protein, notch core proteins, and a further expressible element which can trigger expression of a second effector molecule.

The present application relates generally to a natural killer cell which is derived from a pluripotent stem cell, stability transformed with a synthetic notch receptor protein construct, said construct comprising, nucleic acid domain segments which encode for, reading from 5′ to 3′; a signal peptide, TIGIT protein, notch core proteins, said notch core domain comprising a series of 3 LNR elements, said notch core domain further sequentially linked to an expressible ADAM/TACE element that is linked to a Ga14-VP64 expressible element. It is further provided that the TIGIT protein may be a TIGIT protein or fragment thereof, or more particularly a TIGIT scFv. It is also provided that the described notch core protein element can be functionalized utilizing the ADAM/TACE element or other suitable substitutes which result in downstream activation of genetic elements.

The disclosure covers a method for modifying NK cell activity comprising transforming a pluripotent stem cell of NK cell progenitor cell with an expression construct encoding for a TIGIT/CD155 binding protein, notch core protein activation component and an expressible Ga14-VP64 element.

In some illustrative embodiments, the present disclosure relates to a transformed natural killer cell as described above where said synthetic notch receptor protein construct is further linked to a secondary effector molecule construct which encodes for a UAS, signal peptide and effector molecule.

In some illustrative embodiments, the present disclosure relates to a cell as described above where said secondary effector molecule is an anti-CD73 binding protein. It is a further embodiment of the present disclosure, the transformative vector comprises the DNA sequence of SEQ ID NO: 1, and substantially encodes for the construct depicted in FIG. 9A. A core embodiment is a transformed cell which expresses at the very least a construct comprising the sequence of SEQ ID NO: 2.

In some illustrative embodiments, the present disclosure relates a cell as described above where said secondary effector molecule is an anti-CD137 (4-1BB; 41BB) binding protein.

In another illustrative embodiment, the transformed natural killer cell has more than one expressible secondary effector molecule encoding construct expressibly linked.

In some illustrative embodiments, the present disclosure relates to a method for altering target cell function, where said target cell has CD155 expression, said method comprising contacting said target cell with a natural killer cell which has been transformed as described above, for sufficient time so as to alter the cell function of said target cell.

A method as described above, further comprising administering an effector molecule which blockades CD137 (4-1BB) on said target cell, in sufficient amount and for sufficient time so as to alter the cell function of said target cell.

In some illustrative embodiments, the present disclosure relates to a method as described above, further comprising administering an effector molecule which blockades CD137 (4-1BB) on said target cell, in sufficient amount and for sufficient time so as to alter the cell function of said target cell.

In some other illustrative embodiments, is a method as described above, wherein said effector molecule is an anti-CD137 (4-1BB) antibody or binding fragment thereof.

In a further illustrative embodiment, the modified iNK cells will have prolonged activity in the presence of target cells expressing CD155, as compared with un-modified NK cells. In a further embodiment, the target cells also express CD73. In another further embodiment, co-administration of anti-CD137 blockading molecule enhances the prolonged activity of the iNK cells. An example of such a construct would be exemplified by the sequence of SEQ ID NO: 1.

It is further envisioned that the present disclosure describes transformed NK cells which express a construct that comprises at least the operational domains of an TIGIT/CD155 binding protein, notch protein, and signal domains which would release a triggering molecule that activates the expression of the secondary effector molecule construct, either linked to the TIGIT targeting construct or separately transformed. It is provided for by this disclosure, NK cells transformed with a construct comprising at least the sequence of SEQ ID NO: 2. One of ordinary skill in the art would know and understand how to use standard molecular biology tools, cell expression tools and the like, to make modifications and changes that would be within the scope of this disclosure.

In a further embodiment, the prolonged activity of iNK cells in the presence of target cells expressing CD155 will result in comparatively more killing of target cells, than unmodified NK cells. In a further embodiment, the target cells also express CD73. In another further embodiment, co-administration of anti-CD137 blockading molecule enhances the target cell killing activity of the iNK cells.

Provided is a method for modulating NK cell suppression by a target cell; said method comprising modifying pluripotent stem cells with a transforming vector to generate a transfected Natural Killer cell (iNK), said transforming vector a synthetic notch receptor protein (synthetic notch protein; synNotch) construct which targets the TIGIT/CD155 interaction and optionally provides for subsequent activation of an additional effector molecule which interacts with the cell surface of the target cell. The present disclosure provides for exposing the iNK to a target cell expressing CD155, and optionally additional target molecules such as CD73 and CD137 (41BB); in combination with a blockade molecule which blockades the additional target molecule from function. It is further provided that this blockade molecule is a monoclonal antibody which binds to 41BB or CD73. It is further provided that the additional molecule can be a binding fragment of a monoclonal antibody, or a single chain binding protein (scFv) with specificity for the additionally expressed target molecule or the like.

While the disclosure provides for a single expression construct which encodes for one or more expressible protein including the effector molecule such as anti-CD73. Also provided for is the construct of an expression vector targeting TIGIT/CD155 interaction further comprising the nucleic acid sequence of SEQ ID NO. 3. Also provided is where the iNK is additionally transformed with a second expression vector where expression of a bioactive molecule is affected by the Ga14-VP64 or other similar enzymatic effector, that is released by the synthetic notch protein transformation vector, such a vector is exemplified by a construct comprising a nucleic acid sequence of SEQ ID NO. 3. It is also provided that the additional effector molecule released by the second expression vector encodes for an 4-1BB or CD73 binding molecule. In addition, the disclosure provides for more than one additional effector molecule expression vector. It is provided that the additional effector molecule expressed from the second expression vector interacts with the CD73 on the target cell. It is further provided that this CD73 effector molecule is an antibody derived binding fragment which binds to CD73 (svCD73). Such further effector molecule constructs may be independent of linked to the TIGIT targeting construct and would contain at minimum a construct as exemplified by the sequence of SEQ ID NO: 3.

It is also provided that the secondary effector molecule may be a chemical, pharmaceutical product, binding protein, or other such biologically active molecules which are suitable for modulating target cell activity, including responses such as death, lysis, apoptosis, homeostasis, inhibition, inactivation, shrinkage and the like.

It is also provided that the binding of the additional effector molecule to a cell surface molecule on the target cell, may be about 99% specific for the target molecule. Specificity may range from about 100% to 80% specific for the target molecule. Specificity in some instances can be anywhere from about 50% to 80%, or about 75% to 95%. In other embodiments it is provided that specific for secondary effector molecule may be from about 5% to about 50%.

It is also provided that the iNK of the disclosure could be utilized in combination with other chemical or pharmaceutical oncogenic, anti-cancer type molecules, drugs and pharmaceuticals to enhance the effectiveness of such treatment.

It is also provided that the modulation of inhibition of the iNK cell in contact with a target cell can range from 5% less inhibition as compared to untransformed NK. This can range from about 2% to 50% less inhibition; or from 2% to about 25% less inhibition. In some cases anywhere from about 50% to about 90% less inhibition may occur.

An additional object provided for are iNK cells which functionally express the synthetic notch receptor protein construct (FIG. 4) which target the TIGIT/CD155 interaction. It is an object of the disclosure to provide for the therapeutic and pharmaceutical use of said iNK cells.

It is provided for the pharmaceutical use of the disclosed iNK would be useful to effect natural killer cell activity against target cells expressing CD155 while modulating the suppressive effect of the target cell on the NK cells. The resultant inhibition or killing of target cells would be improved over that of utilizing untransformed NK cells by about 5% to about 25% as measured by conventional means. The provided for inhibition or killing of target cells can range from about 10% to 90% improved activity as compared with nontransformed NK cells. Such pharmaceutical preparations will be dependent upon the mode of administration and patient specific factors similar to that used for any cell therapy and/or monoclonal antibody treatment. It is provided that the natural killer cells are the iNK cells described herein. It is provided that the administration of the iNK to a GBM cell will reduce suppression of the NK cell activity. It is provided that the co-administration of the iNK to GBM with a blockade of 4-1BB will reduce the suppression of NK cell activity. It is further provided that the co-administration of the iNK and 41BB blockade molecule to GBM; with an additional CD73 binding effector molecule will reduce suppression of the NK cell activity. It is provided that suppression of NK cell activity may be monitored by various methods.

The present disclosure provides for a method for altering target cell function, where said target cell has CD155 expression, said method comprising contacting said target cell with a natural killer cell which is derived from a pluripotent stem cell, stability transformed with a synthetic notch receptor protein construct, said construct comprising, nucleic acid domain segments which encode for, reading from 5′ to 3′; a signal peptide, TIGIT binding protein, notch core proteins, said notch core domain comprising a series of 3 LNR elements, said notch core domain further sequentially linked to an expressible ADAM/TACE element that is linked to a Ga14-VP64 expressible element, for sufficient time so as to alter the cell function of said target cell.

More generally the present disclosure provide for a method for altering target cell function, where said target cell has CD155 expression, said method comprising contacting said target cell with a natural killer cell which is derived from a pluripotent stem cell, stability transformed with a synthetic notch receptor protein construct, said construct comprising, a TIGIT/CD155 binding protein, a notch core protein domain and functional activation domain such as ADAM/TACE element that is linked to a expressible element which can further activate a secondary effector molecule expression, such as Ga14-VP64, for sufficient time so as to alter the cell function of said target cell.

### BRIEF DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken into conjunction with the accompanying drawings.

Embodiments of the present disclosure will now be described by way of example in greater detail with reference to the attached Figures, in which:

FIG. 1 shows CD16 (left) and 4-1BB (right) expression induced by TIGIT mAb blockade (50 μg/mL) in NK cells cocultured with GBM43 cells at an E:T ratio of 2.5:1.

FIG. 2 shows Sorted NK cells expressing high levels of TIGIT exhibit enhanced lysis of GBM43 cells in vitro, as well as increased degranulation (CD107a) and cytokine secretion (IFN-γ).

FIG. 3 shows blockade of 4-1BB (10 μg/mL) and TIGIT (50 μg/mL) enhances NK cell cytolysis of GBM43 cells in vitro.

FIG. 4 shows Sequence (top) and diagram (bottom) of TIGIT-synNotch and anti-CD73 genetic constructs.

FIG. 5 shows gene expression (left: %, right, MFI) of electroporated NK cells engineered with anti-CD73 and TIGIT blocking mRNA.

FIG. 6 shows Cytotoxicity (left), CD107a expression (middle) and (IFN-γ) secretion (right) of engineered NK cells against GBM43 cells.

FIG. 7 illustrates the construct of the present disclosure; an “If-gated” synthetic notch protein (TIGIT.CD73) genetic construct structure. This synthetic notch protein genetic construct consists of a “sensing” element which expresses TIGIT linked to intracellular signaling (LNR-Lin-12 Notch repeats; ADAM/TACE—protease cleavage site). Protease activity activates the transcription factor Ga14-VP64, in turn activating the “response” element which triggers the release of the effector molecule.

FIG. 8A data shows cytotoxicity of iPSC-NK cells (iNK) against GBM43 cells upon co-blockade of TIGIT and CD73. *p<0.05; FIG. 8B shows data for expression of the synNotch TIGIT-CD73-Ga14-VP64 gene on iPSC-NK cells (green). >60% expression obtained after iPSC-NK sorting and culture.

FIG. 9A-FIG. 9L depicts data that shows that engineered iNK cells are phenotypically similar to non-engineered cells throughout differentiation and expansion. iNK cells generated via a feeder-free differentiation protocol can be engineered to stably express a synNotch-based genetic construct. (FIG. 9A) Diagram of TIGIT.synNotch.aCD73 genetic construct structure consisting of TIGIT-synNotch-gal4VP64 recognition domain and UAS-aCD73 inducible scFv. (FIG. 9B) Schematic diagram of TIGIT.synNotch.aCD73 activation and subsequent secretion of aCD73 scFv directly at the NK:cancer interface. FIG. 9C Gene expression percentage (left) and MFI (right) of TIGIT.synNotch.aCD73 on iNK cells following differentiation measured via mCherry expression via flow cytometry. FIG. 9D TIGIT expression percentage (left) and MFI (right) measured on TIGIT.synNotch.aCD73-engineered iNK cells via flow cytometry. (FIG. 9E) Purity of CD34+, CD34+CD43+, and CD34+CD45+ cells following 11 days of hematopoietic progenitor differentiation. (FIG. 9F) Representative dot plots of data depicted in FIG. 2E. (FIG. 9G) Engineered iNK cell purity following 39 days of NK cell differentiation. (FIG. 9H) Engineered iNK cell purity following 39 days of NK cell differentiation and an additional 2 weeks of iNK cell expansion. (FIG. 9I, FIG. 9K) Flow cytometry phenotype of engineered iNK cells following 39 days of NK cell differentiation as well as following 2 additional weeks of iNK cell expansion. (FIG. 9J, FIG. 9L) Fold expansion and viability of engineered iNK cells for 10 days following NK cell differentiation.

FIG. 10A-FIG. 10M depicts data that shows that engineered iNK cells, engineered with an inducible genetic construct can target CD155 and CD73-expressing patient-derived GBM cells. Dual targeting of CD73 and CD155 enhances iNK cell activity against CD73+/CD155+ GBM in vitro. (FIG. 10A) Cytotoxicity of iNK cells with or without mAb blockade of TIGIT and/or CD73 against GBM43-WT primary patient derived GBM target cells. (FIG. 10B) Activation of TIGIT.synNotch.aCD73 genetic construct following overnight coculture with GBM43-WT or GBM43 CD155/CD73 KO cells detected via GFP expression via flow cytometry. (FIG. 10C-I) Cytotoxicity of engineered iNK cells versus WT iNK against GBM targets: GBM43 WT, GBM43 CD155 KO, GBM43 CD73 KO, GBM43 CD155/CD73 KO, SJ-GBM2, GBM10, and U87-MG. (FIG. 10J-K) CD107a expression of iNK cells following 4 hour coculture with GBM43 WT target cells. (FIG. 10L) IFN-γ expression by engineered iNK cells following 4 hours of coculture with GBM43 WT target cells. (FIG. 10M) TNF-α secretion by engineered iNK cells following 24 hours of coculture with GBM43 WT target cells. *p<0.05

FIG. 11A-FIG. 11I depicts data that shows that dual targeting of CD73 and CD155 dramatically slows tumor growth in an immunocompetent mouse model. Dual blockade of CD73 and CD155 enhances anti-tumor immune responses in immunocompetent GBM mouse model. (FIG. 11A) Study timeline of GL261 tumor-bearing mice treated with or without CD73 and/or CD155 mAb. (FIG. 11B-C) Tumor volume and bodyweight measurements of mice measured throughout the course of the study. (FIG. 11D) Percentage of various immune cells measured on circulating CD45+ cells in immunocompetent mice measured via flow cytometry. (FIG. 11E) Phenotypic analysis of circulating NK and T cells isolated from treated mice measured by flow cytometry. (FIG. 11F) Percentage of various immune cells measured on splenic CD45+ cells in immunocompetent mice measured via flow cytometry. (FIG. 11G) Phenotypic analysis of splenic NK and T cells isolated from treated mice measured by flow cytometry. (FIG. 11H) Percentage of various immune cells measured on tumor-infiltrating CD45+ cells in treated immunocompetent mice measured via flow cytometry. (FIG. 11I) Phenotypic analysis of tumor-infiltrating NK and T cells isolated from treated mice measured by flow cytometry. *p<0.05 **p<0.01, ***p<0.001, ****p<0.0001

FIG. 12A-FIG. 12G depicts data that shows that Co-targeting CD73 and CD155 with genetically-engineered iNK cells enhances anti-tumor response over targeting either ligand alone. TIGIT.synNotch.aCD73-iNK cells mediate enhanced tumor control over single-targeting iNK cells. (FIG. 12A) Study timeline of GBM43 WT tumor-bearing mice treated with or without non-engineered iNK cells or iNK cells engineered to express either a TIGIT.synNotch, aCD73, or TIGIT.synNotch.aCD73 genetic construct. (FIG. 12B-C) Tumor volume and bodyweight measurements of mice measured throughout the course of the study. (FIG. 12D) Tumor weight of GBM43 WT tumors harvested from iNK or PBS-treated mice. (FIG. 12E) Quantification of NK cell infiltration in GBM43 WT tumors. (FIG. 12F-G) Fold MFI and percentage of NK receptor expression measured on iNK cells harvested from treated GBM43 WT tumors. *p<0.05 **p<0.01, ***p<0.001, ****p<0.0001

FIG. 13A-FIG. 13N depicts data that shows that the synthetic notch protein-engineered iNK cells are highly active against GBM in an orthotopic, patient-derived xenograft model in immunodeficient mice. Usurping CD155 and CD73 activities via synNotch activation of iNK cells can nearly halt tumor growth in an orthotopic patient-derived GBM mouse model. (FIG. 13A) Study timeline of GBM43 tumor-bearing mice treated with or without engineered iNK cells. (FIG. 13A B-C) Bioluminescence imaging of GBM43 WT tumor-bearing mice and measurement of bioluminescence intensity for individual mice throughout the course of the study. (FIG. 13A D-E) Average bioluminescence intensity and bodyweight measurements of mice measured throughout the course of the study. (FIG. 13A F) Kaplan-Meier survival plot of PBS, WT iNK or synNotch-engineered iNK-treated tumor-bearing mice. (FIG. 13A G-N) Representative IHC staining (200× magnification) of GzB, NKp46, CD73, and CD155 in whole brain sections harvested from tumor-bearing mice treated with PBS, WT iNK or synNotch-engineered iNK cells, and quantification/scoring of IHC staining. *p<0.05 **p<0.01, ***p<0.001, ****p<0.0001

FIG. 14 shows the DNA sequence of the TIGIT.synNotch.aCD73 construct (SEQ ID NO: 1).

FIG. 15 shows the DNA sequence of the TIGITscFv-synNotch-Gal4 domains (SEQ ID NO: 2).

FIG. 16 shows the DNA sequence of a secondary effector element construct, UAS-Signal-antiCD73 (SEQ ID NO: 3).

FIG. 17 shows the DNA sequence of a TIGIT-scFv (SEQ ID NO: 4).

### DETAILED DESCRIPTION

For the purposes of promoting and understanding of the principles of the present disclosure, reference will now be made to the embodiments illustrated in the drawings, and specific language will be used to describe the same. It will nevertheless be understood that no limitation of the scope of this disclosure is thereby intended.

The present disclosure describes a stable genetically engineered natural killer cell (iNK). Specifically, we have engineered NK cells to target immunosuppression induced by TIGIT on NK cells. In addition, a secondary effector molecule is triggered which targets CD73, a cancer associated enzyme that produces extracellular adenosine, which inhibits NK cell functions. We have extensively studied our NK cells in vitro to understand the functional underpinnings of TIGIT-induced immunosuppression. TIGIT is known in the art, commercial sources are available. We have also shown that we can engineer NK cells with our genetic construct via mRNA electroporation, targeting TIGIT and CD73, and that these engineered NK cells exhibit enhanced function to non-engineered cells.

TIGIT, a receptor expressed on activated NK cells, contributes to such immunosuppression primarily through interactions with CD155. Yet, TIGIT blockade monotherapy has yielded disappointed NK cell responses in solid tumors. Part of this stems from, on one hand, the broad expression of TIGIT on activated and functionally mature NK cells and, on the other hand, the complex receptor-ligand network that TIGIT is involved in, necessitating impairing its inhibitory activities rather than merely blocking its binding capacity.

Despite possessing a formidable innate ability to eliminate foreign targets, natural killer cells fall prey to dysfunction in the microenvironment of GBM. As a consequence, immunotherapies and chemotherapies for GBM have had minimal improvements on patient survival in the past several decades. While many of the pathways of dysfunction have included a shift in the balance of surface receptors toward inhibition via upregulation of receptors such as NKG2A and PD-1/PD-L1, soluble factors—including metabolites, oncometabolites and immunosuppressive cytokines—also play contributing roles by redirecting the metabolism of NK cells toward one that is impermissible to effective anti-tumor responses. One such axis that contributes to NK cell dysfunction involves the cancer-associated poliovirus receptor CD155 and its cognate ligand TIGIT, expressed on NK cells. As a stress-related protein, CD155 has multiple binding partners on the lymphocyte side, namely TIGIT, DNAM-1 and CD96, but it is its interaction with TIGIT that has been shown to contribute the most substantial inhibitory signaling on NK cells. This is partly owing to the expression of an immunoreceptor tyrosine-based inhibitory motif in the cytoplasmic portion of TIGIT, which stimulates immunosuppression upon binding to CD155 on GBM cells. Yet, TIGIT monotherapy has so far resulted in divergent NK cell responses and has not been effective in enhancing CD8+ T cell responses, controlling tumor growth, or enhancing NK cell function in certain scenarios. Because co-expression of TIGIT correlates to other inhibitory receptors on immune cells its apparent role as a co-receptor has most often benefited from dual blockade approaches. On NK cells, this has translated into the requirement of co-activation via IL-15 stimulation or agonism of 4-1BB (CD137). However, in some studies, 4-1BB stimulation neither enhanced cellular degranulation nor improved IFN-gamma production.

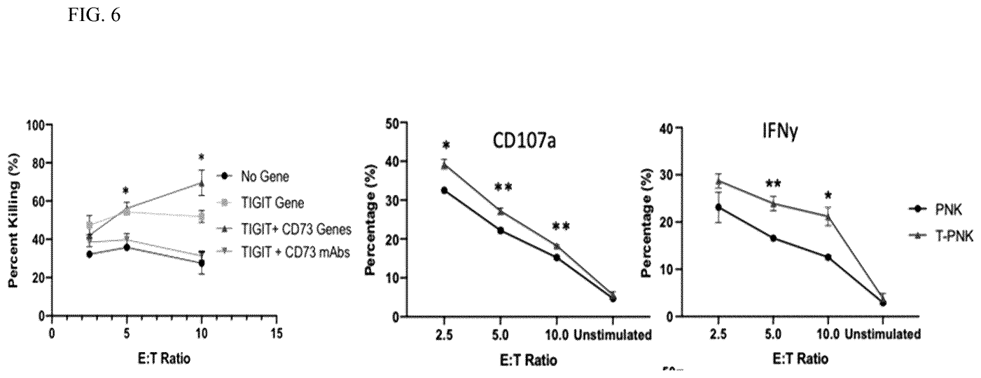

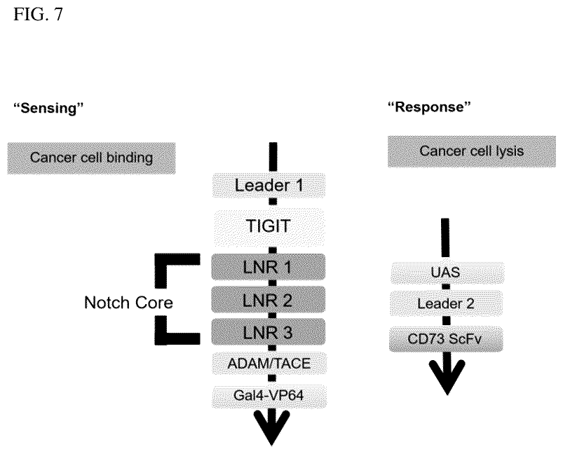

We have developed synthetic notch protein-engineered NK cells which have the capability to block inhibitory binding between TIGIT and CD155. In on example, our construct, TIGIT/ligand engagement, via TIGIT scFv binding to CD155 on GBM, leads to activation of the regulatory region of Notch, followed by a protease cleavage site, which then releases the transcription factor Ga14-VP64 (SEQ ID NO: 2). Transcriptional activation in turn triggers the translation of the and release of CD73 ScFv from the cell (SEQ ID NO: 3). (FIG. 7) The unique aspect of releasing CD73 scFv, rather than a CAR (Chimeric Antigen Receptor), is its ability to target CD73 on cells in the TME that contribute to GBM pathology but are not GBM themselves.

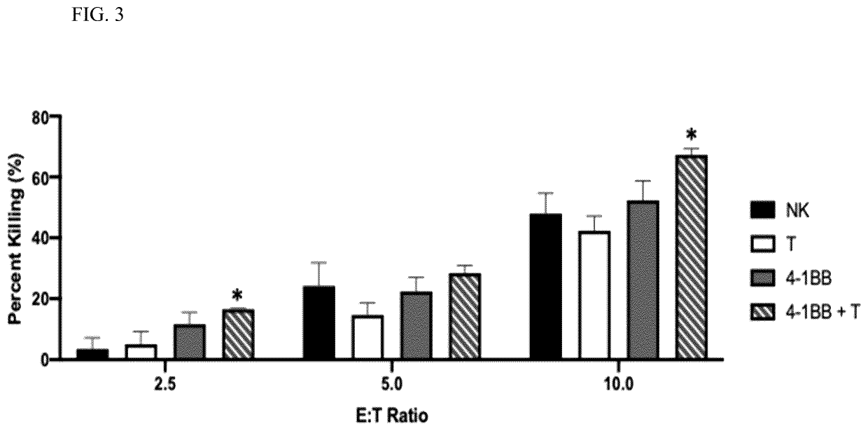

The construct of the present disclosure is based on two different genetic fragments (FIG. 3), which work in tandem to inhibit NK cell immunosuppression. When engaged, the binding of CD155 and TIGIT induces a conformational change in the synNotch protein exposing a cleavable region, which is cleaved by TACE (TACE/ADAM; Tumor necrosis factor-alpha converting enzyme) and release GAL4-VP64. This transcription factor translocates intracellularly, binding with an upstream activating sequence (UAS) on the second gene fragment, and inducing translation and release of an anti-CD73 scFv (derived from MEDI9447) directly to the NK-tumor interface. This system, thereby, blocks immunosuppressive interactions between CD155 and TIGIT and inhibits the generation of immunosuppressive extracellular adenosine by CD73.

In the present disclosure, we have generated from pluripotent stem cells a transformed natural killer cell population (iNK) which retains function of the synthetic notch protein construct, as well as additional effector molecule vector constructs. This iNK cell line is suitably stable for expanded replication, propagation and additional modification. This iNK cell is suitably adapted for targeted interaction to cells with appropriate cell surface components.

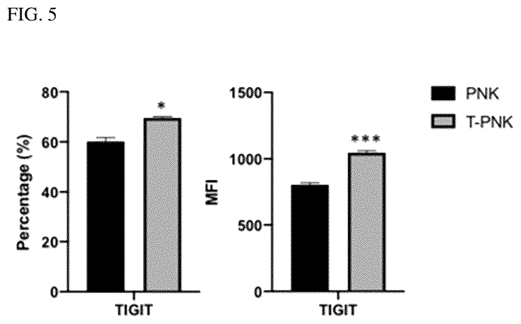

Treatment with engineered iNK cells markedly improved survival over non-engineered NKs and control mice, demonstrating the potency of the CD73/CD155 co-targeting engineered NK cell therapy in treating GBM (FIG. 6F). Thus the present disclosure provides for therapeutic use of the iNK cells disclosed herein.

Mice treated with TIGIT.SynNotch.aCD73 iNK cells had higher numbers of tumor-infiltrating NKp46+ and GzB+NK cells, demonstrating not only a higher infiltration of engineered NK cells into GBM brains, but a higher functional activation compared to WT iNK cells (FIG. 6 G-J). Further, IHC samples were scored for CD73 and CD155 expression, and TIGIT.SynNotch.aCD73 iNK-treated mice exhibited lower levels of CD155 and CD73 than PBS or WT iNK-treated mice, demonstrating the ability of this genetic targeting to function in vivo and effectively downregulate both CD155 and CD73 within the tumor microenvironment (FIG. 6 K-N). Overall, these data show the therapeutic efficacy of a localized dual-blockade of CD155 and CD73 within a synNotch activation cascade directly at the tumor-NK cell interface, and offer a novel potential therapeutic strategy for treating GBM patients.

Accordingly, the disclosure encompasses the use of our TIGIT.SynNotch construct and transformed NK cells in combination with 4-1BB blockade, which results in desired NK cell activity in the GBM tumor micro environment. Due to the apparent relationship between TIGIT and 4-1BB, another inhibitory NK cell receptor, we predict that dual blockade of both TIGIT and 4-1BB may be an efficacious strategy in enhancing NK cell immunotherapies in vivo. The present disclosure teaches the engineering of NK cells to responsively co-block TIGIT and CD73 in the tumor microenvironment. Within this context, we have developed a new approach aimed at curbing TIGIT modulated immunosuppression, via a genetic construct designed to usurp TIGIT activity and its interactions with CD155 on NK cells. The construct is based on a synthetic notch protein signaling system, which triggers transcriptional activation of downstream translation machinery to release a secondary effector molecule, a blocking scFv in one example, in the GBM TME. The net result is the shifting of TIGIT-CD155 immunosuppression toward NK cell activation without directly blocking its binding capacity. Such a “decoy” receptor engages in CD155 binding but usurps the natural progression of this interaction, instead yielding GBM cell killing. This overcomes a major limitation of TIGIT immunotherapy—the ability of CD155 to engage in alternative inhibitory functions upon blockade of TIGIT. The present disclosure utilizes engineered NK cells in combination with 4-1BB blockade.

The present disclosure provides for using induced pluripotent stem cell (iPSC)-derived NK (iNK) cells as allogeneic effectors as a first example of responsively-engineered, dual-targeting iNK cells for a solid tumor.

The present disclosure provides for new immunotherapeutic targets for NK cell therapy in GBM and establish efficacy in targeting them using innovative engineered human NK cells. The disclosure also provides for genetically engineer novel, responsive, multi-specific NK cells against GBM. We have recently discovered a correlation between expression of TIGIT, 4-1BB, and CD16, which suggests that, while inhibitory, high TIGIT expression could actually correlate to more cytolytic NK cells. This understanding is paramount for efficient NK cell engineering and targeting of solid tumors and could potentially enhance clinical success of NK cell therapies as a whole. Further, this proposal is significant in that it is the first report of concomitant targeting of CD73-induced immunometabolic suppression and CD155-TIGIT-induced NK inhibition and presents a promising strategy for targeting GBM.

The following examples serve to illustrate the present disclosure. The examples are not intended to limit the scope of the claimed invention in any way.

### Example 1

We recently characterized TIGIT as a heterogeneous receptor on NK cells in GBM, fueled by evidence of its role in NK cell maturation and sensitivity to inhibitory interactions driven by CD155. (FIG. 7) In turn, this led to our demonstration that effectiveness of TIGIT immunotherapy requires the shifting of TIGIT interactions away from immunosuppression involving its ligands, rather than the mere blockade of TIGIT.

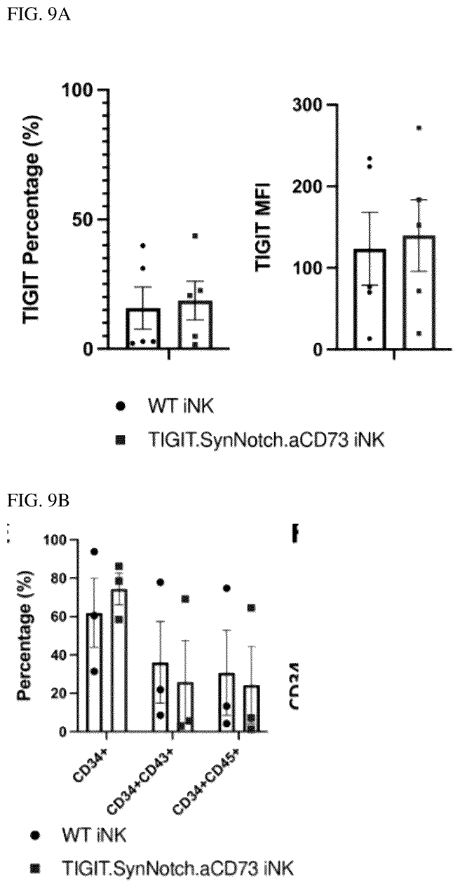

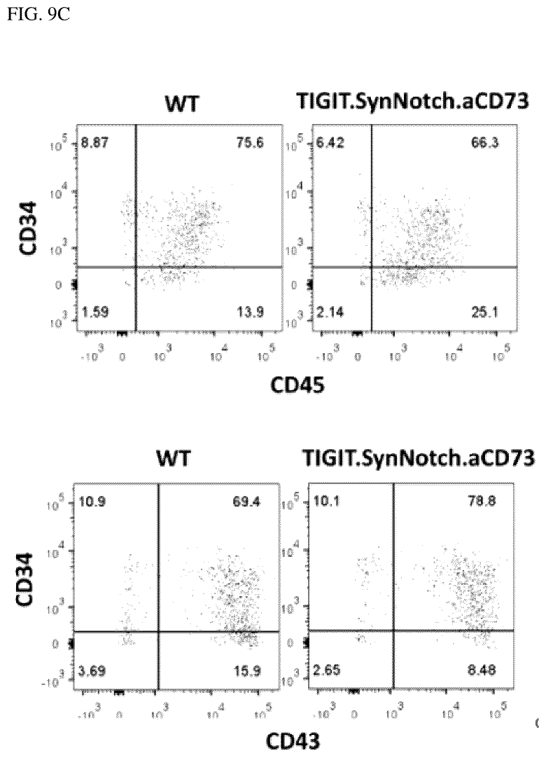

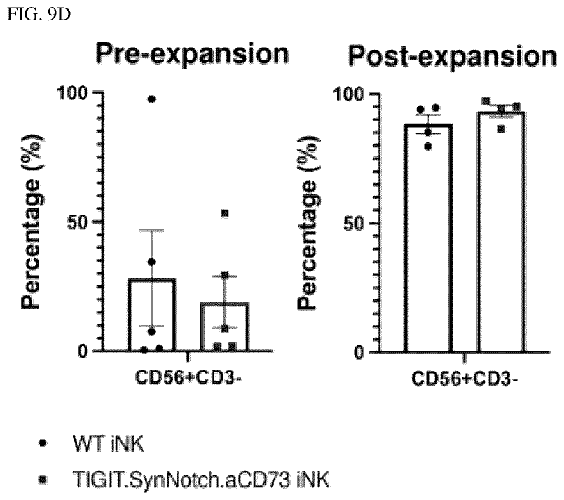

To achieve such selective “role reversal” of TIGIT and combine effective ablation of the CD155-TIGIT axis while promoting NK cell metabolic activity in GBM, we developed a synNotch-based (Synthetic Notch Receptor; Synthetic Notch Protein) construct that responds to CD155 as a priming antigen to induce transcriptional activation that results in release of an antibody to block immunometabolic activity in GBM, in this case via adenosine-producing CD73. (FIG. 9A) These genetically modified NK cells recognize and bind CD155 via a TIGIT extracellular ligand, the binding of which to CD155 not only blocks immunosuppressive TIGIT/CD155 interactions, but also promotes GAL4-VP64 transcription factor-mediated binding to an upstream activating sequence within the construct, to spur the translation and release of an anti-CD73 scFv directly within the GBM tumor microenvironment (TME). Such a construct acts as a “decoy” system by usurping TIGIT-CD155 interactions and in turn promoting NK cell killing of GBM. We previously showed that local blockade of CD73 activity, via a cleavable antibody fragment, is an efficient way of not only suppressing inhibitory concentrations of adenosine in the GBM TME, but also promoting NK cell immunometabolic activity against this tumor.

Usurping the TIGIT-CD155 axis via overexpression of a synNotch construct on NK cells not only takes over inhibitory signaling by redirecting TIGIT activity toward immune-promoting Notch-activation, but it also precludes TIGIT from binding to its other binding partner, CD112, to induce secondary signaling on NK cells in the tumor micro environment (TME), as well as the involvement of CD155 in other inhibitory interactions otherwise possible with TIGIT blockade alone. It also avoids antigenic downregulation of CD155 on GBM cells as a means of “antigen escape” by masking TIGIT-ITIM as a stimulatory receptor.

### Example 2

The present disclosure encompasses using induced pluripotent stem cell (iPSC)-derived NK (iNK) cells as allogeneic effectors. These iPSC-derived NK cells are a potent cellular platform with the potential for allogenicity. Using these cells, generated under chemically-defined, feeder-free conditions and readily amenable to xeno-free manufacturing processes, we describe the first example of engineered allogeneic iPSC-derived NK cells for GBM immunotherapy. By using iPSC-derived NK cells, we have addressed two key challenges associated with cell-based immunotherapy for GBM: poor ex vivo expansion and genetic manipulation of primary NK cells, and limitations associated with functional impairment of autologous NK cells. Synthetic Notch Receptor Protein-based (SynNotch-based) activation of NK cells against GBM via TIGIT achieves the local blockade of CD155 and CD73, directly within the GBM TME, thereby mitigating any off-tumor effects and antigen escape, and maximizing the efficacy of targeting GBM-associated checkpoints, by delivering locally-blocking ligands and antibody fragments directly to the GBM-NK cell interface.

As a first example of responsively-engineered, dual-targeting iNK cells for a solid tumor the disclosure describes iNK which target the TIGIT/CD155 interaction and subsequently enable activation of a secondary effector molecule. These engineered iPSC-NK cells induced remarkable anti-GBM responses in patient-derived orthotopic xenografts, and can be manufactured at scale while sustaining the TIGIT synthetic notch protein expression. In this example, we achieve antigen recognition of CD155 by engineering iPSCs with synthetic notch protein genetic constructs, and subsequently differentiating these cells into functional NK cells. Our finding of a therapeutic correlation between TIGIT and CD73 in GBM, demonstrates the benefit of these two pathways as therapeutic co-targets in GBM, and the superior potential of usurping their activities in contrast to pharmacological blockade alone. Our study paves the way for new clinical immunotherapy treatments, while expanding the current GBM-targeted therapies by characterizing the first-reported multi-functional, inducible engineered iNK cell therapy for treatment of GBM.

Human iPS cell line hiPSCO1 was engineered to express an inducible genetic construct based on the synthetic notch protein signaling system, designed to co-target CD155, via TIGIT, and CD73 (FIG. 9A-B). Briefly, this construct consists of an extracellular TIGIT ligand coupled with a synthetic Notch (synNotch) transmembrane domain, followed by a GAL4-VP64 transcription factor. Binding of TIGIT with CD155 induces a conformational change within the notch domain, revealing a cleavable peptide, which is cleaved by tumor necrosis factor-alpha converting enzyme, or ADAM17 (FIG. 9B). This cleavage releases the GAL4-VP64 transcription factor, which in turn binds to an upstream activating sequence (UAS) on a second genetic construct, initiating the transcription, translation, and release of a second effector molecule, in this example, an anti-CD73 scFv at the GBM-NK cell interface. Fully differentiated iNK cells are able to efficiently express the TIGIT.SynNotch.aCD73 genetic construct following lentiviral, as measured by flow cytometry (FIG. 9C). WT and TIGIT.SynNotch.aCD73 iNK cells express TIGIT at similar levels, allowing us to accurately assess the blocking effect of the TIGIT.SynNotch construct against CD155+ GBM targets (FIG. 9D).

Further, following hematopoiesis, as well as following NK cell maturation and expansion from CD34+ progenitors, iNK cells were assessed for phenotypic markers characteristic of hematopoietic progenitor cells and mature NK cells, respectively. Engineered iNK cells are able to initiate hematopoiesis and maintain expression of markers characteristics of hematopoietic and progenitor cells prior to lineage commitment (FIG. 9E-F). Following NK cell differentiation, engineered iNK cells can be generated at similar purity to WT iNK cells (FIG. 9G). Additionally, after two weeks of NK cell expansion (post-expansion), both engineered and WT iNK cells reach high levels of NK cell purity (>80%) (FIG. 9H).

Engineered iNK cells also express typical NK cell activating and inhibitory markers, at levels similar to those of non-engineered iNK cells both following differentiation and after expansion. Further, engineered iNK cells are able to expand at rates similar to those of non-engineered iNK cells, maintaining high viability throughout expansion.

CD155 and CD73 were selected as targets, due to their overexpression in GBM, and negative roles in patient prognosis. Co-targeting CD155 and CD73 relieves the GBM microenvironment from two major sources of immune resistance: a potent immunosuppressive axis which directly inhibits NK cell function, and metabolic reprogramming of the local TME that directly impacts metabolic fitness. In addition, in vivo inhibition studies showed that co-targeting these two receptors results in favorable reprogramming of the GBM TME, via enhanced recruitment of CD8⁺T cells and concomitant loss of M2 macrophages. This was associated with phenotypic changes on effector immune cells, including a marked downregulation of PD-1 on NK cells. Conversely, targeting either alone has limited therapeutic efficacy. Here we show, through bioinformatics analyses of transcriptional patient data, in vivo efficacy and survival analysis, as well as pharmacological blockade assays, that targeting of CD155/TIGIT and CD73 carries substantial anti-GBM potential, with dramatic preclinical responses. In our experience, synNotch-based engineering of iPSC-NK cells was robust with minimal leakiness, and strongly relied on the presence of targeted antigens on cancer cells. The unique aspect of the iPSC-NK platform is the robustness and relative ease of engineering iPSCs in the undifferentiated state—compared with the routine difficulty at engineering human NK cells—in addition with these cells' ability to retain transduced gene expression throughout hematopoiesis and differentiation into mature immune cells. These engineered iPSC-NK cells are phenotypically indistinguishable throughout all phases of differentiation and expansion yet elicit potent effector functions against CD155±CD73+ GBM cells both in vitro and in vivo. Additional modifications to generate fully allogeneic and patient-compatible, hypoimmunogenic iPSCs are possible through HLA editing.

### Example 3

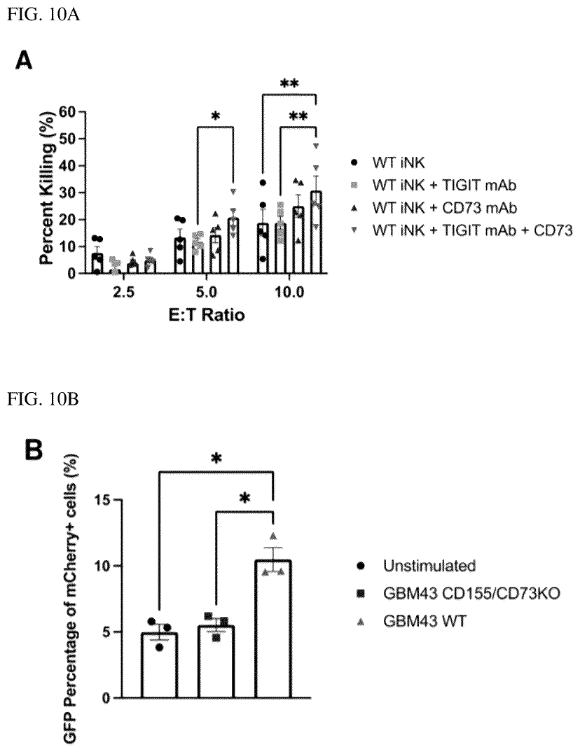

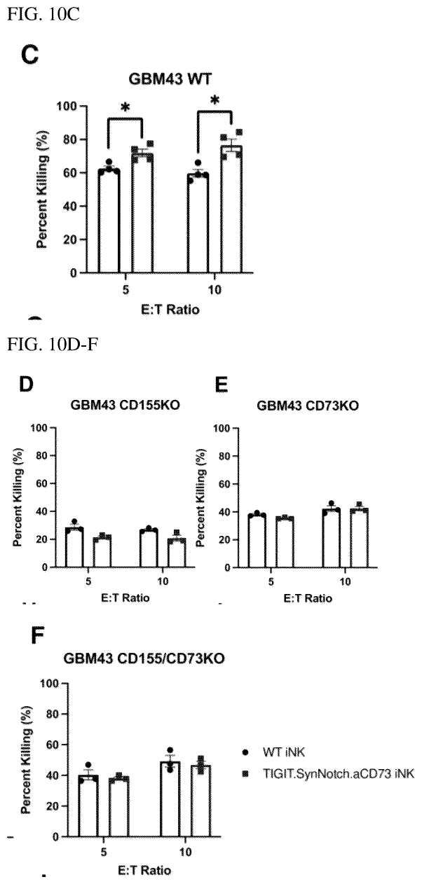

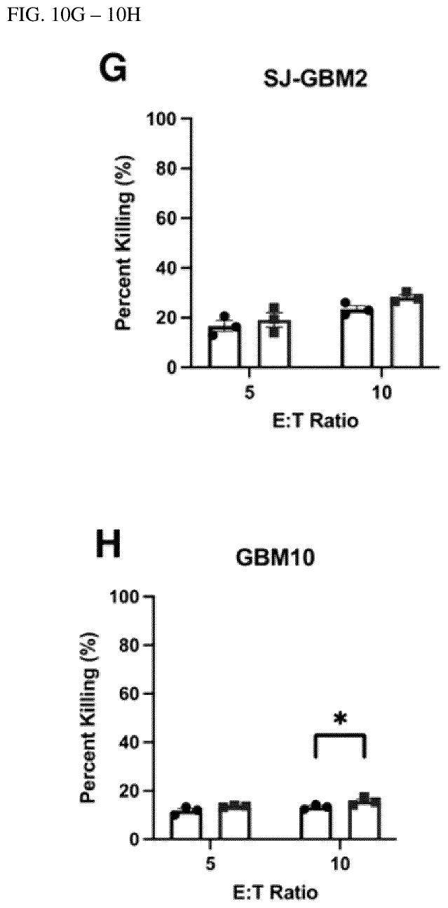

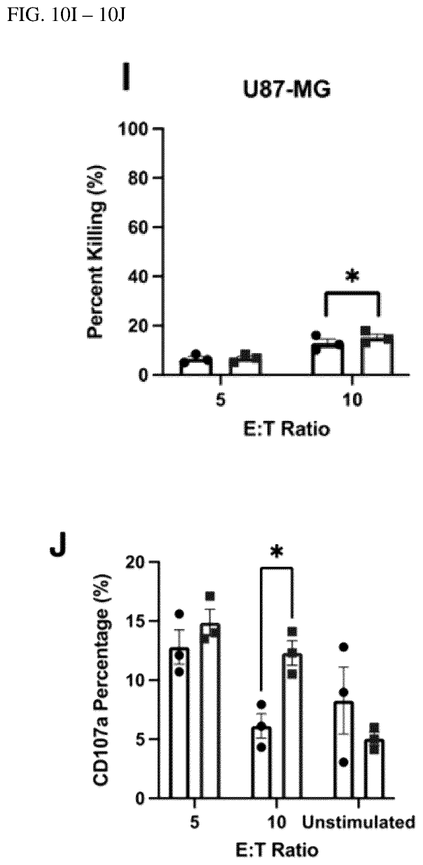

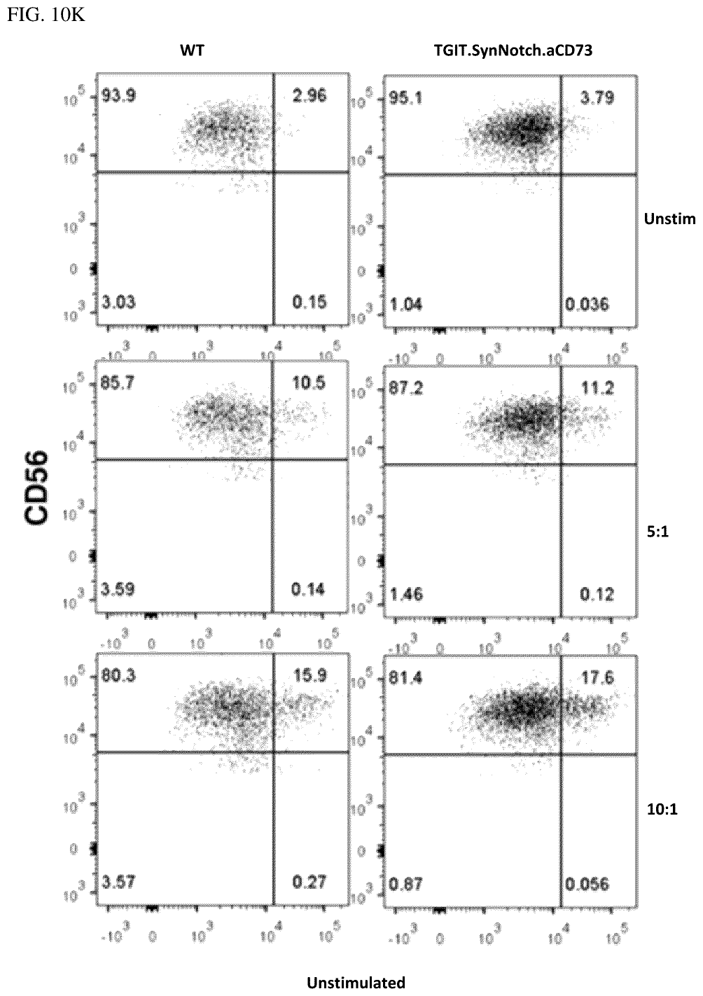

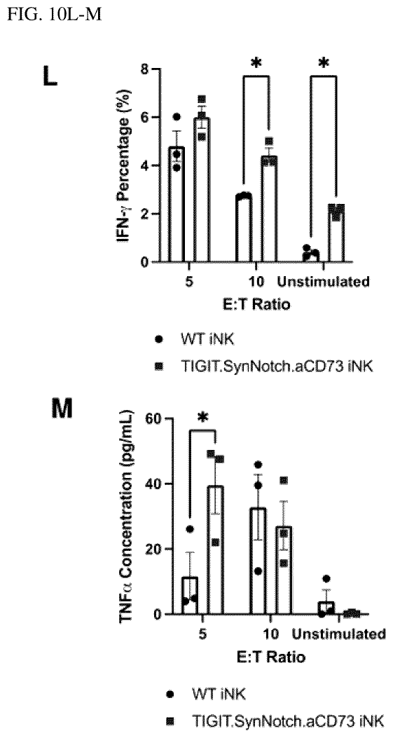

We first assessed the functional efficacy of dual blockade of CD73 and CD155/TIGIT using blocking antibodies. In the presence of both TIGIT and CD73 blocking antibodies, WT iNK cells were able to lyse GBM43 WT cells at significantly higher levels than without antibody blockade. However, either CD73 or TIGIT blockade alone did not significantly enhance the cytolysis capacity of WT iNK cells against GBM43 target cells (FIG. 10A). Activation of the TIGIT.SynNotch.aCD73 construct was assessed against GBM43 WT (CD155+CD73+) target cells and GBM43 CD155/CD73 KO target cells. An increase in GFP expression was measured on mCherry+ TIGIT.SynNotch.aCD73 iNK cells cocultured overnight with CD155+CD73+ GBM43 WT target cells, but not GBM43 CD155/CD73 KO cells, when compared to unstimulated controls, indicating the activation of the aCD73 portion of our construct (FIG. 10B). Engineered iNK cells were then challenged in vitro with GBM cell lines expressing or lacking CD155 (generated via CRISPR/Cas9 knockout) and/or CD73 in cytolysis assays and with GBM43 WT cells for degranulation and cytokine production. TIGIT.SynNotch.aCD73-engineered iNK cells lysed GBM43 WT cells, which express both CD73 and CD155, at significantly higher levels than WT iNK cells (FIG. 10C). However, engineered iNK cells were equivalent to WT iNK cells in lysing GBM cells lacking these receptors, namely CD155KO, CD73KO, and double CD155/CD73KO cell lines, demonstrating the specificity of engineered iNK cells for CD73 and CD155 target ligands (FIG. 10D-F). Additionally, TIGIT.SynNotch.aCD73 iNK cells demonstrated superior lysis against CD155+CD73+ GBM10 and U87-MG targets, but not CD155+CD73-SJ-GBM targets (FIG. 10G-I). These results demonstrated the requirement for the presence of both CD155 and CD73 engagement via the synNotch cascade for optimal activation of iNK cells, and suggesting minimal leakiness of the synNotch construct owing to the inability of engineered iNK cells to lyse targets without proper receptor/ligand activation.

Further, engineered iNK cells challenged with GBM43 WT cells expressed more CD107a and IFN-γ, and secreted more TNF-α than WT iNK cells, further demonstrating the functional enhancement of genetically engineered iNK cells (FIG. 10J-M).

### Example 4

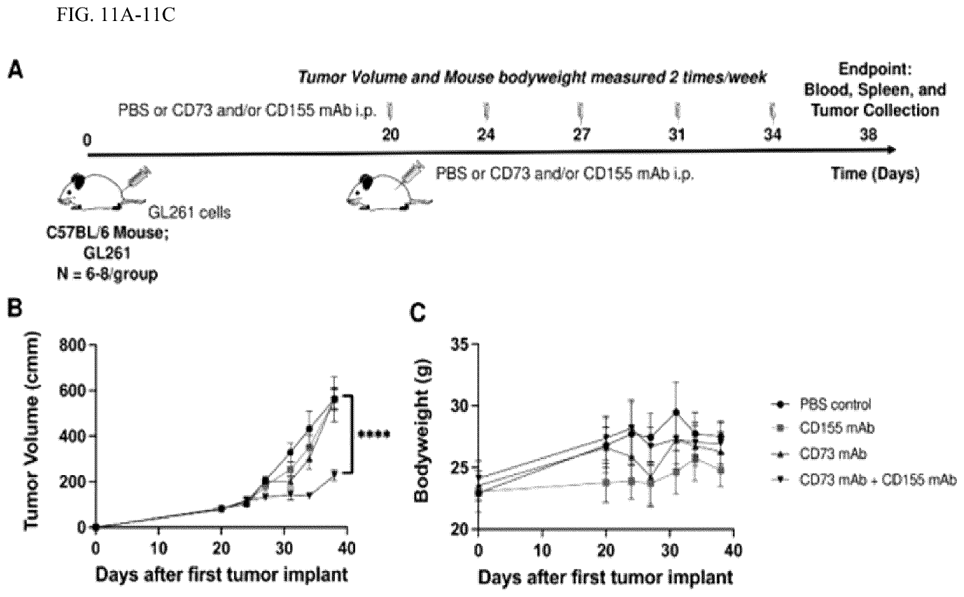

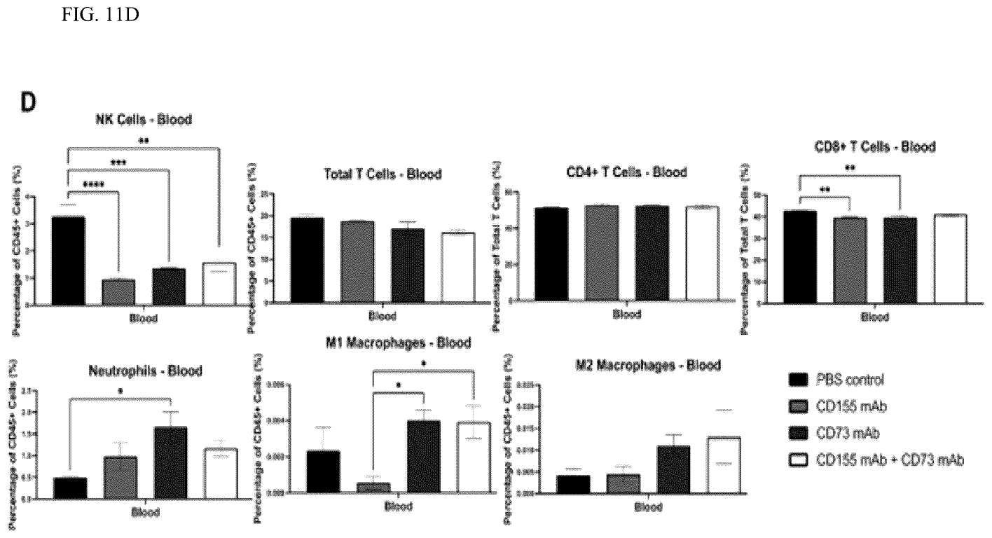

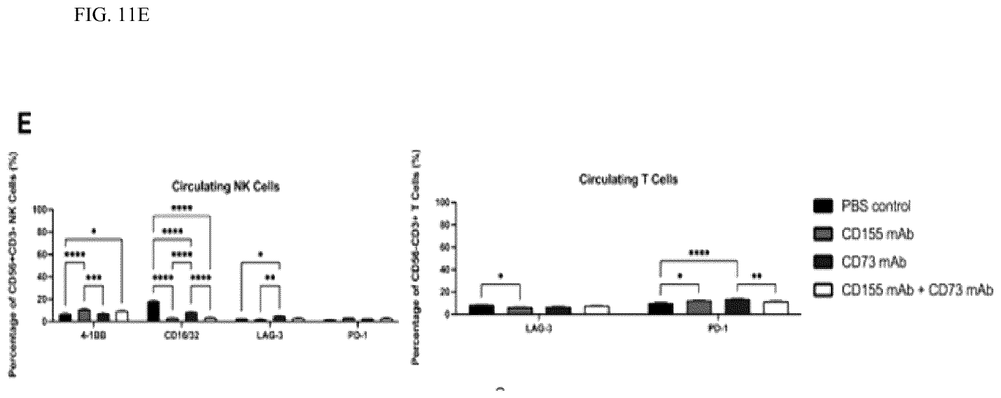

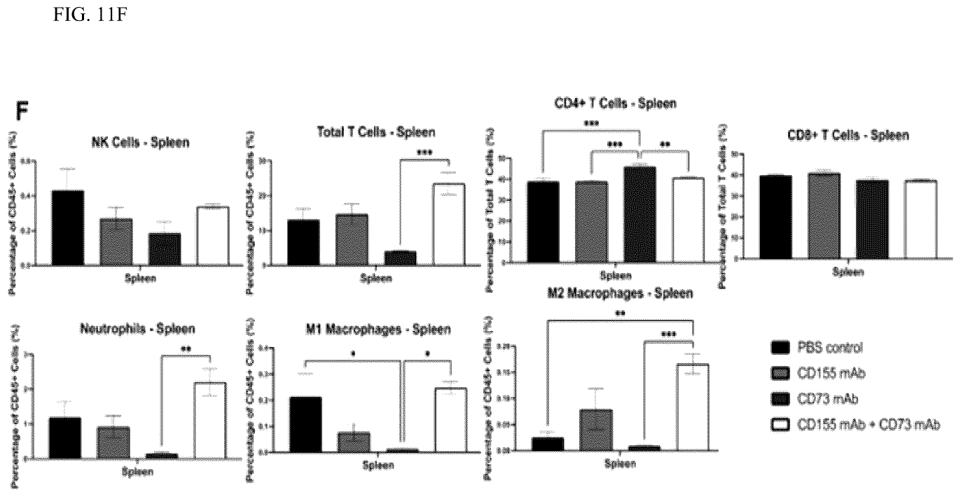

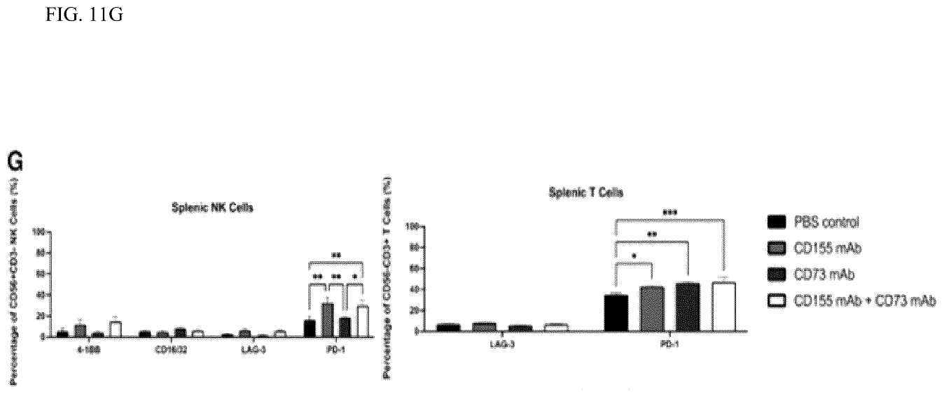

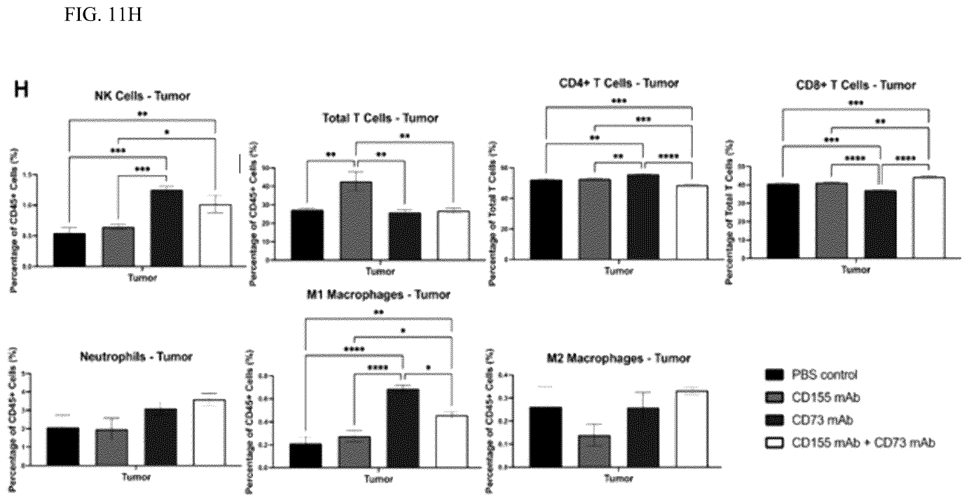

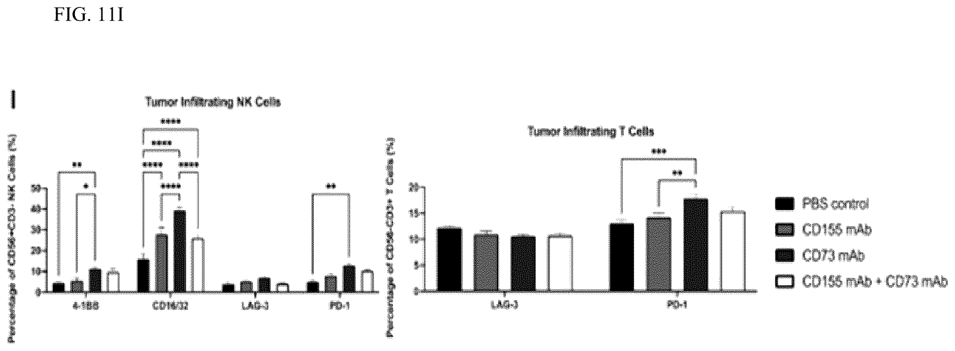

Immunocompetent C57BL/6 mice were treated with or without CD155 and/or CD73 blocking antibodies for 2.5 weeks following engraftment of GL261, mouse glioma, flank tumors (FIG. 11A). Dual treatment with both CD155 and CD73 blocking antibodies significantly slowed tumor growth compared to either antibody alone, or a PBS control group, demonstrating the efficacy of combined CD155 and CD73 targeting (FIG. 11B-C).

To further test co-targeting, we evaluated the cytotoxicity of NK cells against GBM43 WT tumor cells in combination with dual blockade of TIGIT and 41BB and found that such treatment could enhance NK cell lysis of GBM43 tumor cells in vitro. After treatment, immune cells were isolated from the blood, spleen, and tumor tissues of all mice and profiled via flow cytometry (FIG. 11D-I) Immune populations were first distinguished into monocyte, lymphocyte, and granulocyte populations based on CD45 staining, and subsequently separated into the following sub-populations: NK cells, (NK1.1+CD3−), T cells (NK1.1-CD3+), CD4+ T cells, CD8⁺T cells, regulatory T cells (CD4+CD25+FoxP3+), neutrophils (F4/80+Ly6G+), M1 macrophages (F4/80+MHC II+iNOS+), M2 macrophages (F4/80+CD206+Arg-1+). NK and T cells were further phenotyped for activation and exhaustion markers. Notably, there was an upregulation of tumor-infiltrating NK cells and CD8+ T cells in mice treated with both CD73 and CD155 mAbs compared to the PBS control, and a downregulation of CD4+ T cells (FIG. 11H). Mice treated with CD73 mAb alone were shown to have an increase in tumor infiltrating NK cells, but a decrease in CD8⁺T cells. Further, we show an increase in anti-tumor M1 macrophages and no significant change in the pro-tumor M2 macrophage in the tumors of CD73/CD155 dual-treated mice. NK and T cell phenotypes were further analyzed for expression of various inhibitory and activating markers (FIG. 11I). We observed an upregulation of inhibitory PD-1 in both NK and T cells treated with CD73 mAb alone, and no significant upregulation in the dual CD73/CD155 mAb treatment group, indicating that dual targeting of CD73 and CD155 may enhance anti-tumor immune responses and mitigate functional exhaustion of effector immune cells, when compared to single targeting of CD155 or CD73 alone. Although the phenotype of tumor-infiltrating NK cells upon dual CD73/CD155 treatment was comparable to that of NK cells treated with CD155 alone, dual-treated tumors showed a significantly larger proportion of NK cells infiltrating the tumors.

### Example 5

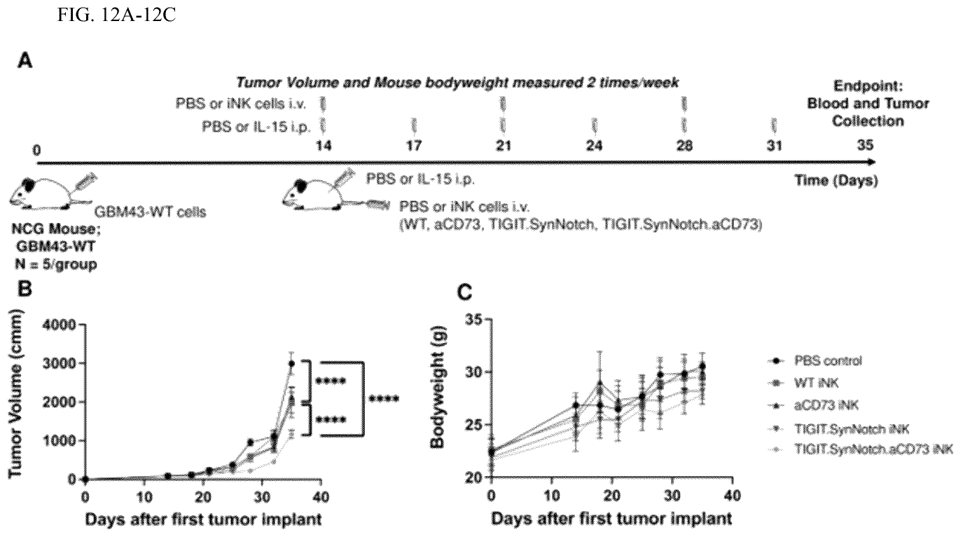

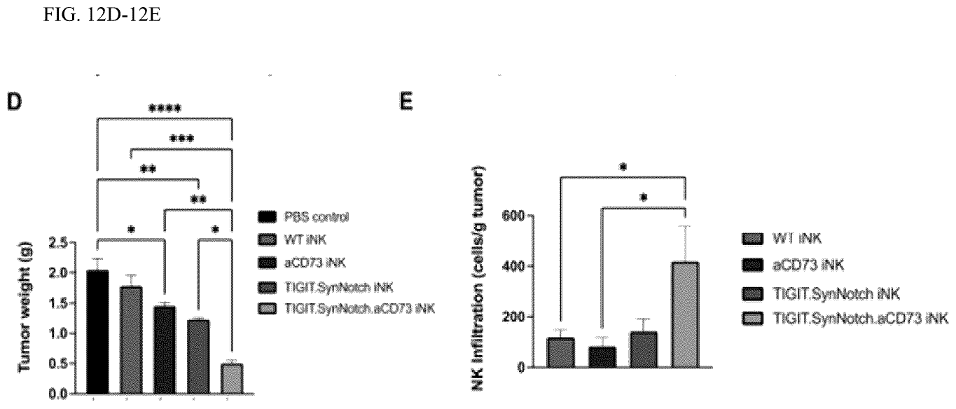

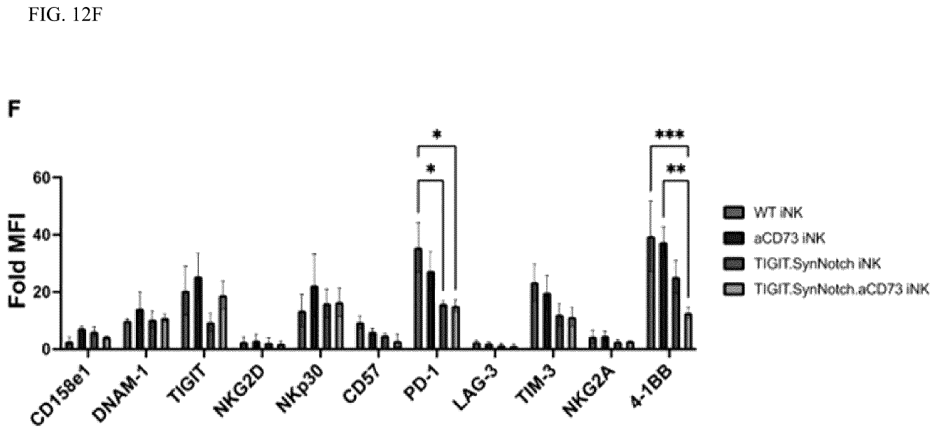

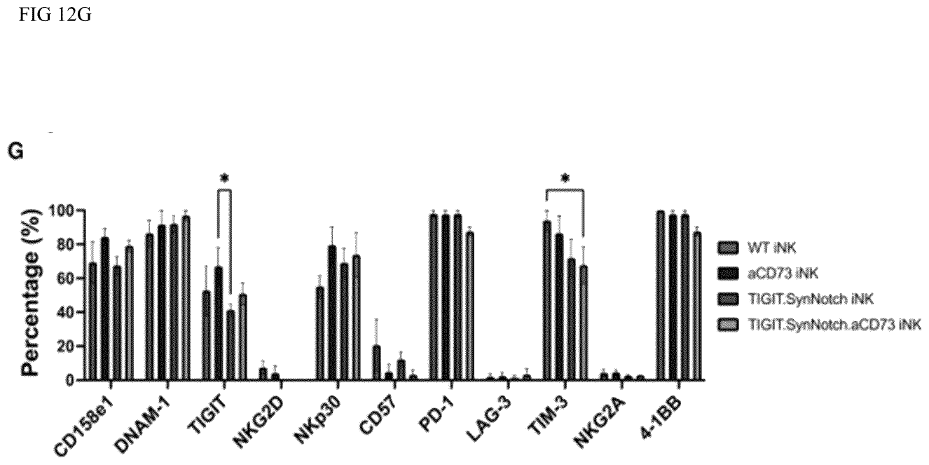

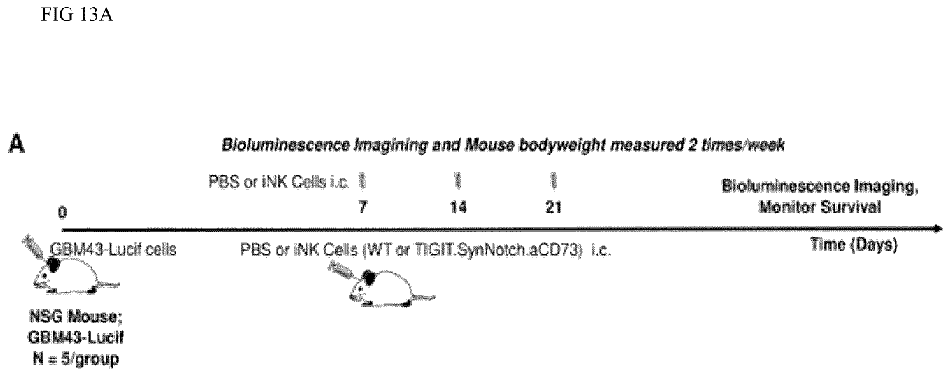

Immunodeficient NCG mice were engrafted with GBM43 WT flank tumors, and, after tumors were established, mice were treated with or without iNK cells according to the following treatment groups: WT, aCD73, TIGIT.SynNotch, and TIGIT.SynNotch.aCD73 (FIG. 12A). Treatment of GBM43 tumors with iNK cells from any group reduced tumor progression over the PBS control group. Further, dual-targeted TIGIT.SynNotch.aCD73 iNK cells demonstrated enhanced anti-tumor responses over either WT or engineered iNK cells lacking the aCD73 domain, demonstrating the value of simultaneous targeting of CD73 and TIGIT in an adoptive transfer setting and the need for the transcriptional activation and translation of CD73-blocking scFv following TIGIT-CD155 engagement (FIG. 12B-C). Tumors harvested from TIGIT.SynNotch.aCD73 NK cell-treated mice showed a significant reduction in tumor weight when compared with the other treatment groups (FIG. 12D). Further, there was an upregulation in the number of infiltrating NK cells in TIGIT.SynNotch.aCD73-iNK-treated mice compared to WT iNK-treated mice, indicating an enhanced activity of dual-engineered iNK cells (FIG. 12E). Tumor-infiltrating NK cells harvested from TIGIT.SynNotch.aCD73-treated mice exhibited a reduction in PD-1, TIM-3, and 4-1BB compared to WT control groups, suggesting that these cells may be more functional and less functionally exhausted than control NK cells (FIG. 12F-G).

### Example 6

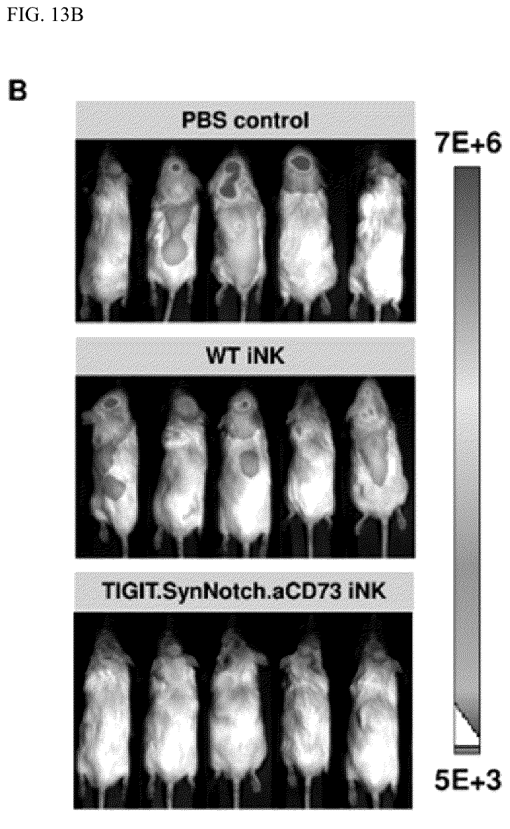

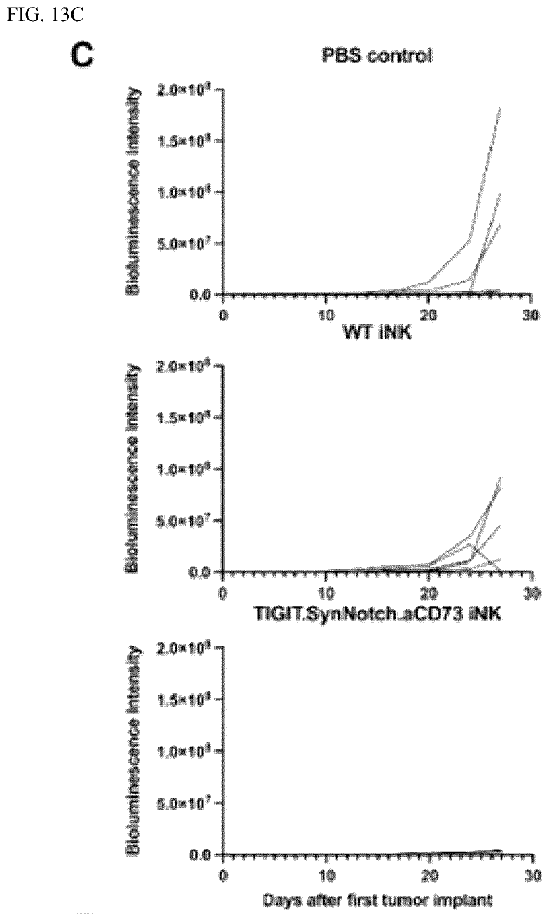

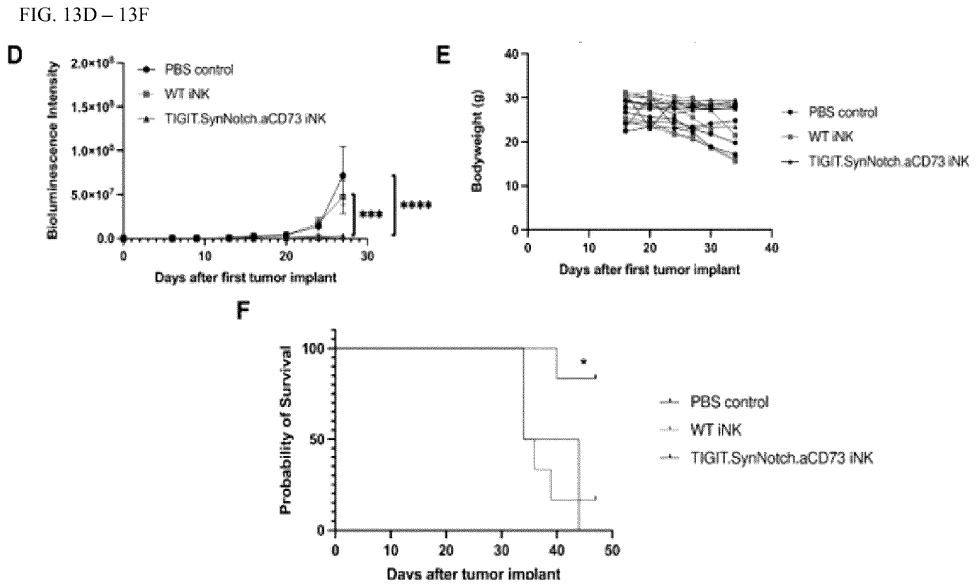

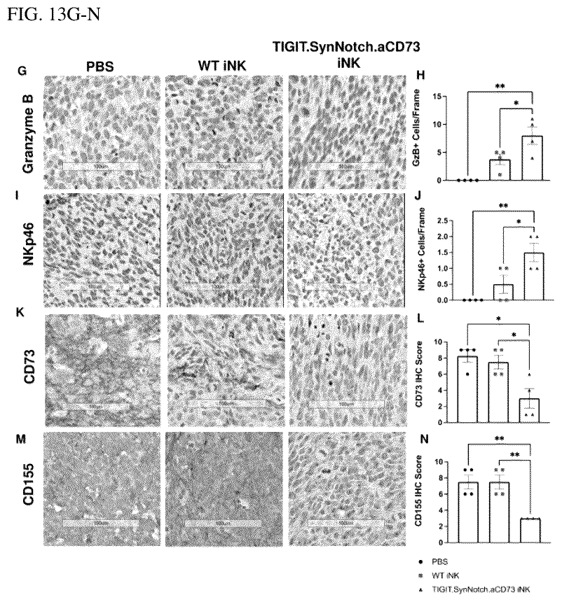

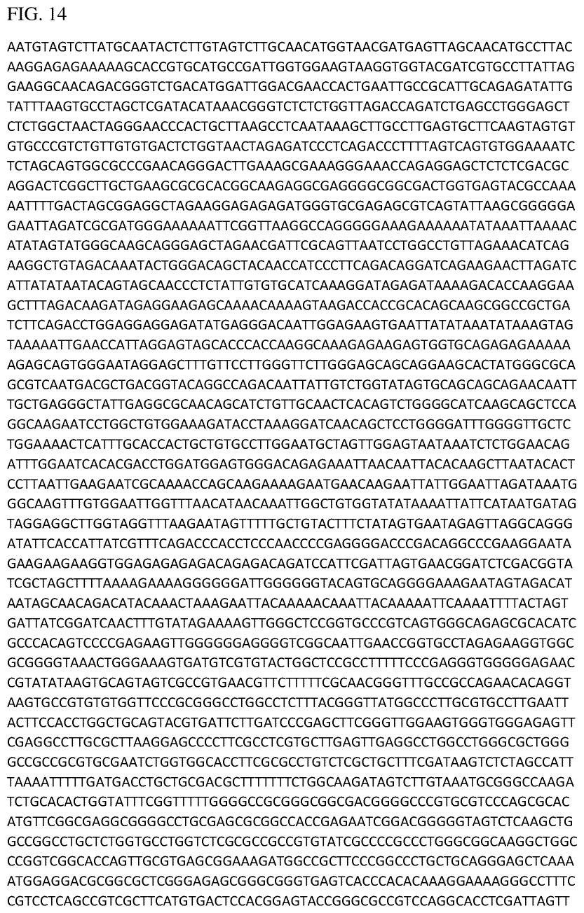

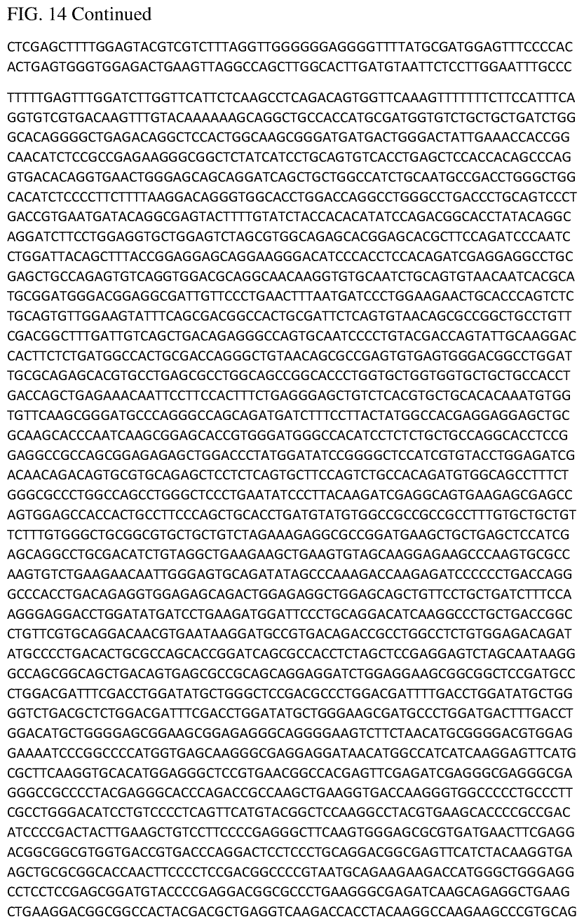

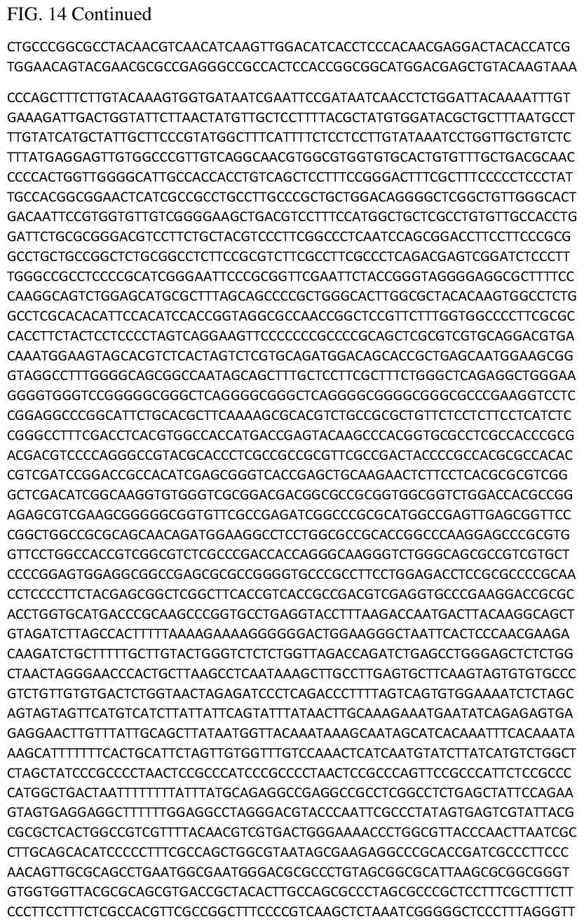

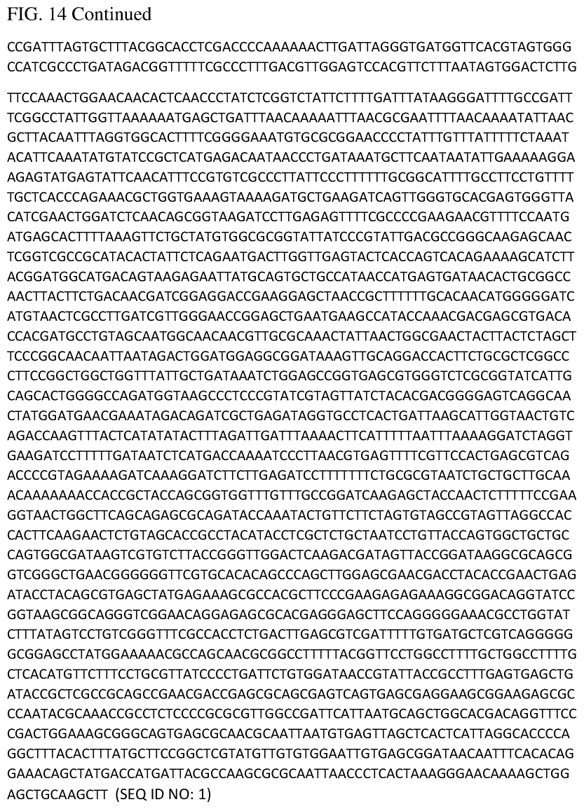

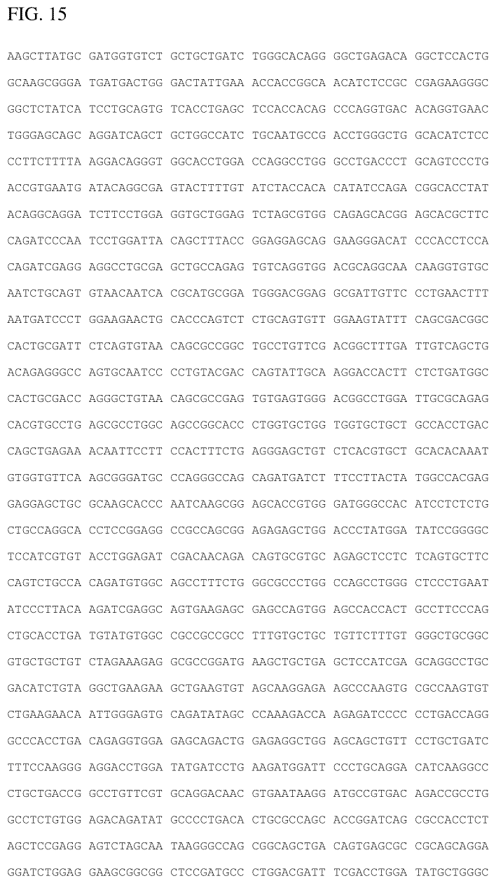

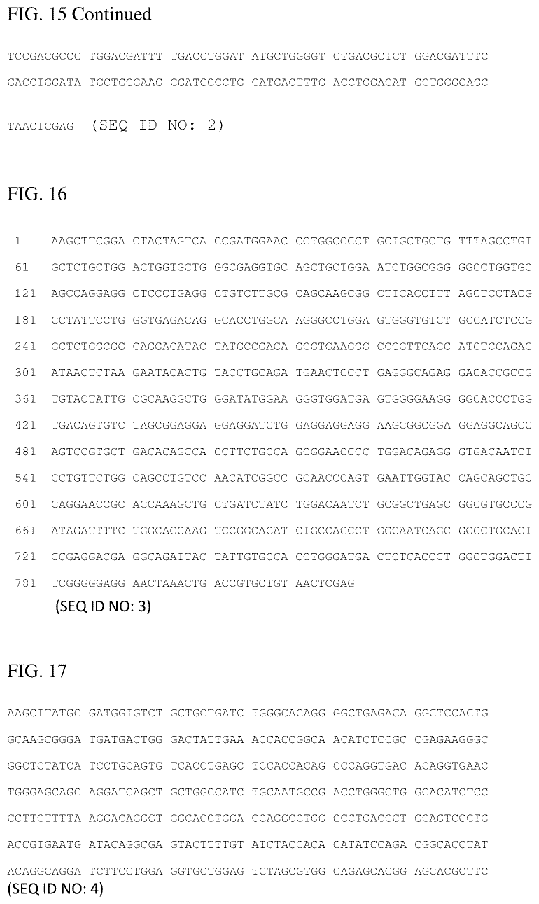

To assess the functional capacity of synNotch-CD73/CD155 co-targeted iNK cells against an intracranial model of GBM, we established a GBM43 xenograft orthotopic model in NSG mice. To do so, we genetically engineered GBM43 cells to express a firefly luciferase gene to enable active monitoring of tumor progression throughout the study. NSG mice were first implanted intracranially with 1×105 GBM43-luciferase cells, then subsequently received three weekly injections of 2×106 WT or TIGIT.SynNotch.aCD73 engineered iNK cells, also intracranially (FIG. 13A). Tumor size and bodyweight were monitored throughout the study (FIG. 13B-E). Mice receiving engineered iNK cells recorded a drastic reduction in tumor growth, when compared with the PBS control group or mice receiving WT iNK cells, with many tumors nearly being eliminated altogether (FIG. 13B-E). Further, treatment with engineered iNK cells markedly improved survival over non-engineered NKs and control mice, demonstrating the potency of the CD73/CD155 co-targeting engineered NK cell therapy in treating GBM (FIG. 13F). Whole brain samples collected from PBS, WT iNK and TIGIT.SynNotch.aCD73 iNK treated mice were harvested and immunohistochemically stained for NKp46, granzyme B (GzB), CD73 and CD155. Mice treated with TIGIT.SynNotch.aCD73 iNK cells had higher numbers of tumor-infiltrating NKp46+ and GzB+NK cells, demonstrating not only a higher infiltration of engineered NK cells into GBM brains, but a higher functional activation compared to WT iNK cells (FIG. 13 G-J). Further, IHC samples were scored for CD73 and CD155 expression, and TIGIT.SynNotch.aCD73 iNK-treated mice exhibited lower levels of CD155 and CD73 than PBS or WT iNK-treated mice, demonstrating the ability of this genetic targeting to function in vivo and effectively downregulate both CD155 and CD73 within the tumor microenvironment (FIG. 13 K-N). Overall, these data show the therapeutic efficacy of a localized dual-blockade of CD155 and CD73 within a synNotch activation cascade directly at the tumor-NK cell interface, and offer a novel potential therapeutic strategy for treating GBM patients.

### Example 7

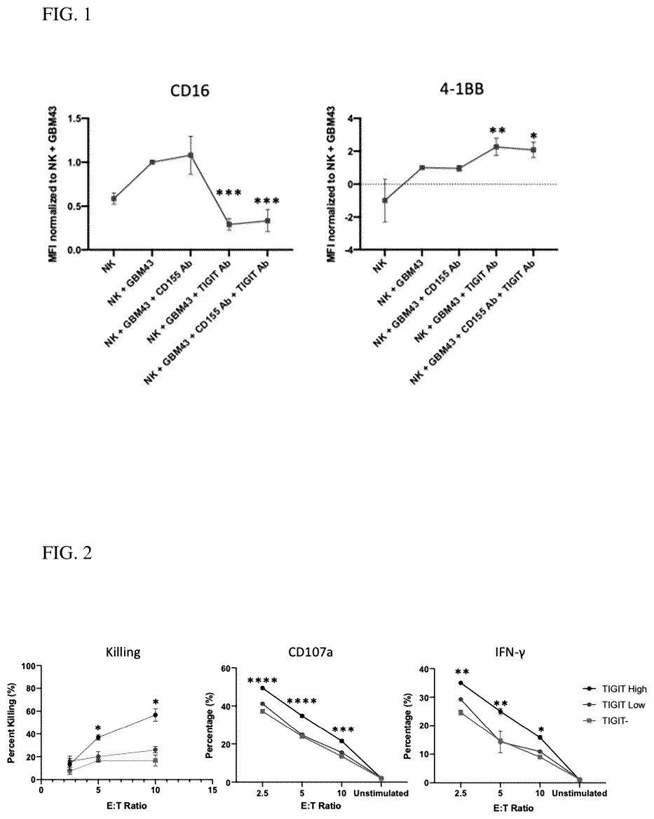

We have cocultured human peripheral blood-derived NK cells with GBM43 cells at an effector:target (E:T) ratio of 2.5:1 for 24 hours with or without TIGIT (50 ug/mL) and/or CD155 (5 ug/mL) mAbs and found, via flow cytometry, that CD16 expression decreases in the response to TIGIT blockade, while 4-1BB expression increases (FIG. 1). Further, we discovered that TIGIThigh NK cells express higher levels of CD16 than either TIGITlow or TIGIT-NK cells. After sorting NK cells based on expression of TIGIT (high, low, negative) we observed that TIGIThigh NK cells were able to lyse GBM43 cells, degranulate, and secrete IFNy at higher levels than either TIGITlow or TIGIT-NK cells. (FIG. 2) This suggests that there may be a relationship between TIGIT, CD16, and 41BB, and that TIGIT, while inhibitory, may play a role in NK cell maturation and development of cytolytic functions. (FIG. 3)

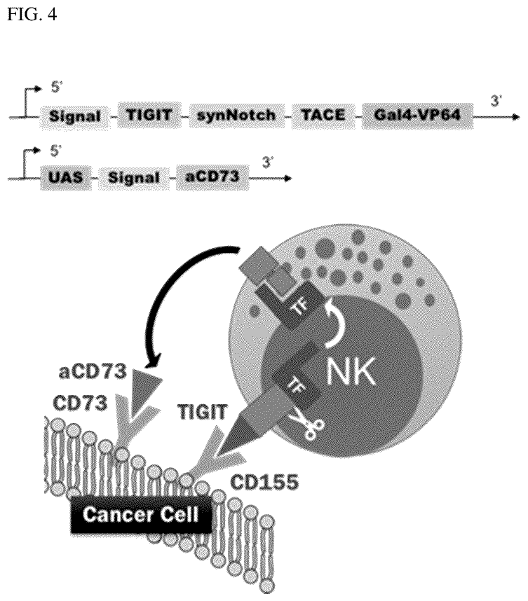

We have also designed and synthesized two different genetic fragments (FIG. 4), which work in tandem to inhibit NK cell immunosuppression. When engaged, the binding of CD155 and TIGIT induces a conformational change in the synthetic notch protein construct, notch protein exposing a cleavable region, which is cleaved by TACE and release GAL4-VP64. This transcription factor translocates intracellularly, binding with an upstream activating sequence (UAS) on the second gene fragment, and inducing translation and release of an anti-CD73 scFv (derived from MEDI9447; US20160129108A1)) directly to the NK-tumor interface. This system, thereby, blocks immunosuppressive interactions between CD155 and TIGIT and inhibits the generation of immunosuppressive extracellular adenosine by CD73. Expanded PNK cells were electroporated with mRNA with the Biorad GenePulser XCell electroporation system using a protocol optimized for our cells and expansion conditions. Gene expression was measured via flow cytometry through TIGIT expression, and we showed that electroporated PNK cells were able to successfully express our genetic construct, as evidenced by an increase in TIGIT expression (FIG. 5). Further, we have demonstrated that our engineered NK cells exhibit superior killing of GBM43 target cells, as well as enhance IFNy secretion and CD107a expression when compared to non-engineered NK cells (FIG. 6).

### Example 8

The construct of the disclosure is based on two different genetic fragments (FIG. 7), which work in tandem to inhibit NK cell immunosuppression. When engaged, the binding of CD155 and TIGIT induces a conformational change in the synNotch protein exposing a cleavable region, which is cleaved by TACE and release GAL4-VP64. This transcription factor translocates intracellularly, binding with an upstream activating sequence (UAS) on the second gene fragment, and inducing translation and release of an anti-CD73 scFv (derived from MEDI9447) directly to the NK-tumor interface. This system, thereby, blocks immunosuppressive interactions between CD155 and TIGIT and inhibits the generation of immunosuppressive extracellular adenosine by CD73

Using a novel responsive genetic construct, we have been able to engineer NK cells to co-target TIGIT and CD73 simultaneously in the local GBM TME. We have shown that these engineered NK cells can enhance in vitro cytolysis of CD73+/CD155+ GBM cells. We believe that this therapy can also enhance anti-tumor activity of NK cells in an in vivo immunodeficient mouse model of patient-derived GBM.

We have shown that blockade of TIGIT and 4-1BB enhances in vitro activity of NK cells against GBM43 cells, a patient-derived primary GBM cell line, (FIG. 1) Based on our data, co-administration of 4-1BB monoclonal antibody (mAb) treatment can further enhance the efficacy of our engineered NK cell therapy in vivo in a glioblastoma mouse model, as a novel combination immunotherapy against GBM or other cancer cells which co-express CD155 and 4-1BB(CD137). (FIG. 3)

### Example 9

We have evaluated our construct with both human and iPSC-NK cells (iNK).

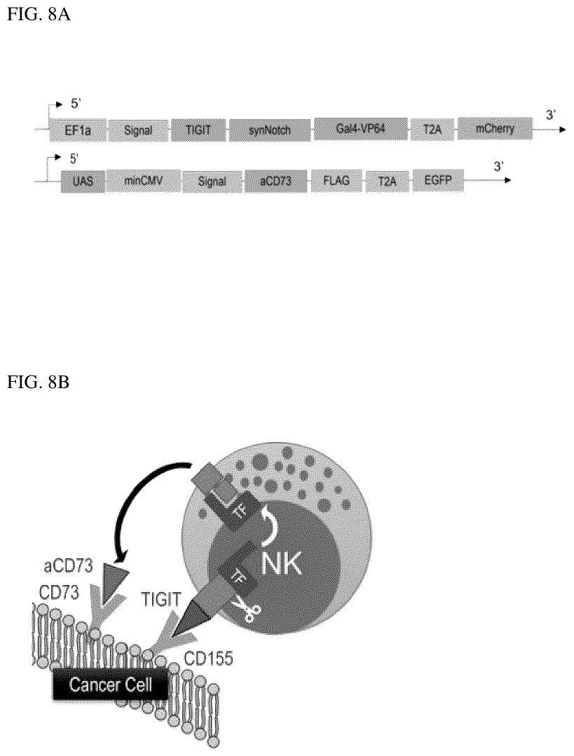

As shown, the iPSC-NK cells (iNK), in particular, present advantages in terms of genetic modification compared to blood NK cells. These are primarily in terms of ease of transducing iPSC-NK cells at the iPSC stage, circumventing resistance to genetic modification characteristic of blood NK cells. We exploited this by demonstrating that iPSC-NK cells are not only responsive to co-blockade of TIGIT+CD73 (FIG. 8A), but they can be engineered to express the synthetic Notch protein construct (FIG. 8B), differentiated to maintain gene expression, and cryopreserved. Synthetic Notch protein-engineered iPSC-NK cells can efficiently mediate cytotoxicity against GBM43 cells. Expanded human NK cells were both electroporated with mRNA with the Biorad GenePulser XCell electroporation system using a protocol optimized for our cells and expansion conditions, and transduced virally with lentivirus encoding our target gene construct. Gene expression was measured via flow cytometry through TIGIT expression, and we showed that electroporated human NK cells were able to successfully express our genetic construct, as evidenced by an increase in TIGIT expression. We also demonstrated that iPSC-NK cells were also able to express the genetic construct. Further, we have demonstrated that our engineered NK cells exhibit superior killing of GBM43 target cells, as well as enhance IFN-γ secretion and CD107a expression when compared to non-engineered NK cells (FIG. 6).

In the present disclosure the term “about” can allow for a degree of variability in a value or range, for example, within 20%, within 10%, within 5%, or within 1% of a stated value or of a stated limit of a range.

In the present disclosure the term “substantially” can allow for a degree of variability in a value or range, for example, within 80%, within 90%, within 95%, or within 99% of a stated value or of a stated limit of a range.

The term “patient” includes human and non-human animals such as companion animals (dogs and cats and the like) and livestock animals Livestock animals are animals raised for food production. The patient to be treated is preferably a mammal, in particular a human being.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffered solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

It is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender, and diet of the patient: the time of administration, and rate of excretion of the specific compound employed, the duration of the treatment, the drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosage may be single or divided, and may be administered according to a wide variety of dosing protocols, including q.d., b.i.d., t.i.d., or even every other day, once a week, once a month, and the like. In each case the therapeutically effective amount described herein corresponds to the instance of administration, or alternatively to the total daily, weekly, or monthly dose.

As used herein, the term “therapeutically effective amount” refers to that amount of active compound or pharmaceutical agent that elicits the biological or medicinal response in a tissue system, animal or human that is being sought by a researcher, veterinarian, medical doctor or other clinicians, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment.

As used herein, the term “therapeutically effective amount” refers to the amount to be administered to a patient, and may be based on body surface area, patient weight, and/or patient condition. In addition, it is appreciated that there is an interrelationship of dosages determined for humans and those dosages determined for animals, including test animals (illustratively based on milligrams per meter squared of body surface) as described by Freireich, E. J., et al., Cancer Chemother. Rep. 1966, (4), 219, the disclosure of which is incorporated herein by reference. Body surface area may be approximately determined from patient height and weight (see, e.g., Scientific Tables, Geigy Pharmaceuticals, Ardley, New York, pages 537-538 (1970)). A therapeutically effective amount of the compounds described herein may be defined as any amount useful for inhibiting the growth of (or killing) a population of malignant cells or cancer cells, such as may be found in a patient in need of relief from such cancer or malignancy. Typically, such effective amounts range from about mg/kg to about 500 mg/kg, from about 5 mg/kg to about 250 mg/kg, and/or from about 5 mg/kg to about 150 mg/kg of compound per patient body weight. It is appreciated that effective doses may also vary depending on the route of administration, optional excipient usage, and the possibility of co-usage of the compound with other conventional and non-conventional therapeutic treatments, including other anti-tumor agents, radiation therapy, and the like. The term “about” as used herein can allow for a degree of variability in a value or range, for example, within 10%, within 5%, or within 1% of a stated value or of a stated limit of a range.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation.

Any use of section headings and subheadings is solely for ease of reference and is not intended to limit any disclosure made in one section to that section only; rather, any disclosure made under one section heading or subheading is intended to constitute a disclosure under each and every other section heading or subheading.

Various modifications and variations of the described compositions, methods, and uses of the technology will be apparent to those skilled in the art without departing from the scope and spirit of the technology as described. Although the technology has been described in connection with specific exemplary embodiments, the invention as claimed should not be unduly limited to such specific embodiments. Indeed, various modifications of the described modes for carrying out the invention that are obvious to those skilled in the art are intended to be within the scope of the following claims.

The terms and expressions, which have been employed, are used as terms of description and not of limitation. In this regard, where certain terms are defined and are described or discussed elsewhere, the definitions and all descriptions and discussions are intended to be attributed to such terms. There also is no intention in the use of such terms and expressions of excluding any equivalents of the features shown and described or portions thereof.

Further, all publications and patents mentioned herein are incorporated by reference in their entireties for all purposes. In the event of inconsistent usages between this document and those documents so incorporated by reference, the usage in the incorporated reference should be considered supplementary to that of this document; for irreconcilable inconsistencies, the usage in this document controls.

We claim:

1. A natural killer cell which is derived from a pluripotent stem cell, stability transformed with a synthetic notch receptor protein construct, said construct comprising, nucleic acid domain segments which encode for, reading from 5′ to 3′; a signal peptide, TIGIT/CD155 binding protein, notch core proteins, said notch core domain comprising a series of 3 LNR elements, said notch core domain further sequentially linked to an expressible ADAM/TACE element that is linked to a Ga114-VP64 expressible element.

2. A cell of claim 1, said construct comprising at least the TIGIT/CD155 binding protein, notch core protein activation component and an expressible Ga14-VP64 element.

3. A cell of claim 2, where said construct comprises the sequence of SEQ ID NO: 2.

4. The cell of claim 1 wherein said synthetic notch receptor protein construct is further linked to a secondary effector molecule construct which encodes for a UAS, signal peptide and effector molecule.

5. The cell of claim 4 wherein said effector molecule is an anti-CD73 binding protein.

6. The cell of claim 4 wherein said construct linked to a secondary effector molecule construct comprises the sequence of SEQ ID NO: 3.

7. The cell of claim 4 wherein said effector molecule is an anti-CD137 (4-1BB) binding protein.

8. A method for altering target cell function, where said target cell has CD155 expression, said method comprising contacting said target cell with a natural killer cell which is derived from a pluripotent stem cell, stability transformed with a synthetic notch receptor protein construct, said construct comprising, nucleic acid domain segments which encode for, reading from 5′ to 3′; a signal peptide, TIGIT binding protein, notch core proteins, said notch core domain comprising a series of 3 LNR elements, said notch core domain further sequentially linked to an expressible ADAM/TACE element that is linked to a Gal14-VP64 expressible element, for sufficient time so as to alter the cell function of said target cell.

9. A method of claim 8, where said construct also encodes for a secondary effector molecule.

10. A method of claim 8, where said construct also encodes for a secondary effector molecule which target is CD73.

11. A method of claim 8, where said construct also encodes for a secondary effector molecule which target is CD137 (4-1BB).

12. A method of claim 8, further comprising administering an effector molecule which blockades CD137 (4-1BB) on said target cell, in sufficient amount and for sufficient time to alter the cell function of said target cell.

13. A method of claim 8, wherein said effector molecule is an anti-CD137 (4-1BB) antibody or binding fragment thereof.

14. A method for modifying NK cell activity comprising transforming a pluripotent stem cell of NK cell progenitor cell with an expression construct encoding for a TIGIT/CD155 binding protein, notch core protein activation component and an expressible Ga14-VP64 element.


SEQUENCE LISTING (4 sequences)

SEQ ID NO: 1 - DNA, 11303 bp, Synthetic construct
  source 1..11303: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgcgatggtgtctgctgctgatctgggcacaggggctgagacaggctccactggcaagcgggatgatgactgggactattgaaaccaccggcaacatctccgccgagaagggcggctctatcatcctgcagtgtcacctgagctccaccacagcccaggtgacacaggtgaactgggagcagcaggatcagctgctggccatctgcaatgccgacctgggctggcacatctccccttcttttaaggacagggtggcacctggaccaggcctgggcctgaccctgcagtccctgaccgtgaatgatacaggcgagtacttttgtatctaccacacatatccagacggcacctatacaggcaggatcttcctggaggtgctggagtctagcgtggcagagcacggagcacgcttccagatcccaatcctggattacagctttaccggaggagcaggaagggacatcccacctccacagatcgaggaggcctgcgagctgccagagtgtcaggtggacgcaggcaacaaggtgtgcaatctgcagtgtaacaatcacgcatgcggatgggacggaggcgattgttccctgaactttaatgatccctggaagaactgcacccagtctctgcagtgttggaagtatttcagcgacggccactgcgattctcagtgtaacagcgccggctgcctgttcgacggctttgattgtcagctgacagagggccagtgcaatcccctgtacgaccagtattgcaaggaccacttctctgatggccactgcgaccagggctgtaacagcgccgagtgtgagtgggacggcctggattgcgcagagcacgtgcctgagcgcctggcagccggcaccctggtgctggtggtgctgctgccacctgaccagctgagaaacaattccttccactttctgagggagctgtctcacgtgctgcacacaaatgtggtgttcaagcgggatgcccagggccagcagatgatctttccttactatggccacgaggaggagctgcgcaagcacccaatcaagcggagcaccgtgggatgggccacatcctctctgctgccaggcacctccggaggccgccagcggagagagctggaccctatggatatccggggctccatcgtgtacctggagatcgacaacagacagtgcgtgcagagctcctctcagtgcttccagtctgccacagatgtggcagcctttctgggcgccctggccagcctgggctccctgaatatcccttacaagatcgaggcagtgaagagcgagccagtggagccaccactgccttcccagctgcacctgatgtatgtggccgccgccgcctttgtgctgctgttctttgtgggctgcggcgtgctgctgtctagaaagaggcgccggatgaagctgctgagctccatcgagcaggcctgcgacatctgtaggctgaagaagctgaagtgtagcaaggagaagcccaagtgcgccaagtgtctgaagaacaattgggagtgcagatatagcccaaagaccaagagatcccccctgaccagggcccacctgacagaggtggagagcagactggagaggctggagcagctgttcctgctgatctttccaagggaggacctggatatgatcctgaagatggattccctgcaggacatcaaggccctgctgaccggcctgttcgtgcaggacaacgtgaataaggatgccgtgacagaccgcctggcctctgtggagacagatatgcccctgacactgcgccagcaccggatcagcgccacctctagctccgaggagtctagcaataagggccagcggcagctgacagtgagcgccgcagcaggaggatctggaggaagcggcggctccgatgccctggacgatttcgacctggatatgctgggctccgacgccctggacgattttgacctggatatgctggggtctgacgctctggacgatttcgacctggatatgctgggaagcgatgccctggatgactttgacctggacatgctggggagcggaagcggagagggcaggggaagtcttctaacatgcggggacgtggaggaaaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 2 - DNA, 2049 bp, Synthetic construct
  source 1..2049: mol_type = other DNA
aagcttatgcgatggtgtctgctgctgatctgggcacaggggctgagacaggctccactggcaagcgggatgatgactgggactattgaaaccaccggcaacatctccgccgagaagggcggctctatcatcctgcagtgtcacctgagctccaccacagcccaggtgacacaggtgaactgggagcagcaggatcagctgctggccatctgcaatgccgacctgggctggcacatctccccttcttttaaggacagggtggcacctggaccaggcctgggcctgaccctgcagtccctgaccgtgaatgatacaggcgagtacttttgtatctaccacacatatccagacggcacctatacaggcaggatcttcctggaggtgctggagtctagcgtggcagagcacggagcacgcttccagatcccaatcctggattacagctttaccggaggagcaggaagggacatcccacctccacagatcgaggaggcctgcgagctgccagagtgtcaggtggacgcaggcaacaaggtgtgcaatctgcagtgtaacaatcacgcatgcggatgggacggaggcgattgttccctgaactttaatgatccctggaagaactgcacccagtctctgcagtgttggaagtatttcagcgacggccactgcgattctcagtgtaacagcgccggctgcctgttcgacggctttgattgtcagctgacagagggccagtgcaatcccctgtacgaccagtattgcaaggaccacttctctgatggccactgcgaccagggctgtaacagcgccgagtgtgagtgggacggcctggattgcgcagagcacgtgcctgagcgcctggcagccggcaccctggtgctggtggtgctgctgccacctgaccagctgagaaacaattccttccactttctgagggagctgtctcacgtgctgcacacaaatgtggtgttcaagcgggatgcccagggccagcagatgatctttccttactatggccacgaggaggagctgcgcaagcacccaatcaagcggagcaccgtgggatgggccacatcctctctgctgccaggcacctccggaggccgccagcggagagagctggaccctatggatatccggggctccatcgtgtacctggagatcgacaacagacagtgcgtgcagagctcctctcagtgcttccagtctgccacagatgtggcagcctttctgggcgccctggccagcctgggctccctgaatatcccttacaagatcgaggcagtgaagagcgagccagtggagccaccactgccttcccagctgcacctgatgtatgtggccgccgccgcctttgtgctgctgttctttgtgggctgcggcgtgctgctgtctagaaagaggcgccggatgaagctgctgagctccatcgagcaggcctgcgacatctgtaggctgaagaagctgaagtgtagcaaggagaagcccaagtgcgccaagtgtctgaagaacaattgggagtgcagatatagcccaaagaccaagagatcccccctgaccagggcccacctgacagaggtggagagcagactggagaggctggagcagctgttcctgctgatctttccaagggaggacctggatatgatcctgaagatggattccctgcaggacatcaaggccctgctgaccggcctgttcgtgcaggacaacgtgaataaggatgccgtgacagaccgcctggcctctgtggagacagatatgcccctgacactgcgccagcaccggatcagcgccacctctagctccgaggagtctagcaataagggccagcggcagctgacagtgagcgccgcagcaggaggatctggaggaagcggcggctccgatgccctggacgatttcgacctggatatgctgggctccgacgccctggacgattttgacctggatatgctggggtctgacgctctggacgatttcgacctggatatgctgggaagcgatgccctggatgactttgacctggacatgctggggagctaactcgag

SEQ ID NO: 3 - DNA, 818 bp, Synthetic construct
  source 1..818: mol_type = other DNA
aagcttcggactactagtcaccgatggaaccctggcccctgctgctgctgtttagcctgtgctctgctggactggtgctgggcgaggtgcagctgctggaatctggcgggggcctggtgcagccaggaggctccctgaggctgtcttgcgcagcaagcggcttcacctttagctcctacgcctattcctgggtgagacaggcacctggcaagggcctggagtgggtgtctgccatctccggctctggcggcaggacatactatgccgacagcgtgaagggccggttcaccatctccagagataactctaagaatacactgtacctgcagatgaactccctgagggcagaggacaccgccgtgtactattgcgcaaggctgggatatggaagggtggatgagtggggaaggggcaccctggtgacagtgtctagcggaggaggaggatctggaggaggaggaagcggcggaggaggcagccagtccgtgctgacacagccaccttctgccagcggaacccctggacagagggtgacaatctcctgttctggcagcctgtccaacatcggccgcaacccagtgaattggtaccagcagctgccaggaaccgcaccaaagctgctgatctatctggacaatctgcggctgagcggcgtgcccgatagattttctggcagcaagtccggcacatctgccagcctggcaatcagcggcctgcagtccgaggacgaggcagattactattgtgccacctgggatgactctcaccctggctggactttcgggggaggaactaaactgaccgtgctgtaactcgag

SEQ ID NO: 4 - DNA, 420 bp, Synthetic construct
  source 1..420: mol_type = other DNA
aagcttatgcgatggtgtctgctgctgatctgggcacaggggctgagacaggctccactggcaagcgggatgatgactgggactattgaaaccaccggcaacatctccgccgagaagggcggctctatcatcctgcagtgtcacctgagctccaccacagcccaggtgacacaggtgaactgggagcagcaggatcagctgctggccatctgcaatgccgacctgggctggcacatctccccttcttttaaggacagggtggcacctggaccaggcctgggcctgaccctgcagtccctgaccgtgaatgatacaggcgagtacttttgtatctaccacacatatccagacggcacctatacaggcaggatcttcctggaggtgctggagtctagcgtggcagagcacggagcacgcttc